(19) United States
(12) Patent Application Publication
(10) Pub. No.: US 2020/0085349 A1
(43) Pub. Date: Mar. 19, 2020

(54) ADHESIVE SYSTEMS HAVING AN AGGRESSIVE ADHESIVE OUTER RING ALONG ITS BOTTOM PERIMETER AND HAVING A LOW EFFECTIVE MODULUS OF ELASTICITY

(71) Applicant: Metronom Health, Inc., Laguna Hills, CA (US)

(72) Inventor: Troy M. Bremer, Irvine, CA (US)

(21) Appl. No.: 16/470,839

(22) PCT Filed: Dec. 21, 2017
(86) PCT No.: PCT/US2017/067824

§ 371 Date: Jun. 18, 2019

Related U.S. Application Data
(60) Provisional application No. 62/439,132, filed on Dec. 26, 2016.

(51) Int. Cl. A61B 5/145

(52) U.S. Cl. CPC A61B 5/14532; A61B 5/1495; A61B 5/14546

(57) ABSTRACT

An adhesive system having a first layer including a first layer material having a top and a bottom having a bottom perimeter, and a first layer adhesive on the bottom for attaching to skin, the first layer having an inherent modulus of elasticity. The adhesive system also includes a second adhesive along only the bottom perimeter, where the first layer includes a plurality of modifications therein that result in the first layer having an effective modulus of elasticity that is lower than the first layer's inherent modulus of elasticity.

### RELATED APPLICATIONS

This application is a National Stage Entry Application of PCT/US2017/067824, filed Dec. 21, 2017, which claims the benefit of U.S. Provisional Application No. 62/439,132, filed Dec. 26, 2016, the entire contents of each of these applications are incorporated herein by reference.

### BACKGROUND

### 1. Field of the Invention

The disclosed and described technology relates generally to single layer and multilayer adhesive systems that can be used to attach, for example, medical devices to skin or can be used, for example, to apply bandages to skin.

### 2. Description of the Related Art

Current adhesive systems have difficulty remaining on the skin for extended periods of time because they do not address the differences in mechanical properties between the skin and the adhesive, i.e., stress/strain differentials that exist between skin and the adhesive systems. Skin typically has a low stress strain relationship that may be approximated as 0.05 MPa for strains of 1.0 or 0.02 MPa for strains of 0.4. The skin is viscoelastic and current adhesive systems are typically highly elastic. Because of the mechanical mismatch between the skin and current adhesive systems, when current adhesive systems are in place on skin and the skin moves (stretches/tension and compresses/compression), these adhesive systems do not move to the same extent as the skin and therefore, experience stress/strain mismatch between the adhesive system material and the skin. This mismatch results in high shear forces at the interface between the adhesive system adhesive layer and the skin upon which it is adhered. As a result of these shear forces, current adhesive systems experience edge peel, which eventually leads to peel off of the entire adhesive system.

Another issue with current adhesive systems is that they suffer from moisture loading (moisture trapped between the skin and the adhesive system) because they have an inadequate moisture vapor transmission rate (“MVTR”), which results in “float off” of the system. MVTR is a measure of the passage of water vapor through a substance and/or barrier. Because perspiration naturally occurs on the skin, if the MVTR of a material or adhesive system is low, this can result in moisture accumulation between the skin and the adhesive system that can promote bacterial growth, cause skin irritation, and can cause the adhesive system to peel away or “float off” from the skin.

Thus, adhesive systems must be designed to (1) address the mismatch of mechanical properties that exist between skin and the adhesive systems and (2) have a high MVTR. Prior adhesive systems have attempted to address the issue of mismatch of mechanical properties and the resulting edge peel, by using aggressive adhesives, i.e., adhesives that have high adhesion to skin. An adhesive's aggressiveness is defined by its initial bond strength and its sustained bond strength. However, these aggressive adhesives do not address the main problem of strain mismatch and the high shear forces that result between the skin and the adhesive and therefore, result in systems that do not expand and contract to the same extent as the skin and remain strongly attached to the skin resulting in very high shear forces leading to pain to the wearer, and which will eventually lead to edge peel and peel off. Additionally, using an aggressive adhesive is very difficult and painful to remove from the skin when a wearer desires to remove the adhesive system. However, an adhesive that is not sufficiently aggressive, will not maintain attachment to the skin as the skin expands and contracts and will result in edge peel and peel off.

Accordingly, adhesive system embodiments of the present invention have been designed to address these deficiencies of prior adhesive systems.

### SUMMARY

Methods and apparatuses or devices being disclosed herein each have several aspects, no single one of which is solely responsible for its desirable attributes. Without limiting the scope of this disclosure, for example, as expressed by the claims which follow, its more prominent features will now be discussed briefly. After considering this discussion, and particularly after reading the section entitled “Detailed Description,” one will understand how the features being disclosed and described provide advantages that include monitoring, diagnosing, and treating a patient using results obtained from an analyte sensor.

Systems are disclosed for an adhesive system for attaching an optical sensor-transmitter system. In one example, the adhesive system is for adhering a medical device to the skin of a patient. The adhesive system can include an outside layer, wherein the outside layer is elastic and re-sealable to the skin of the patient. The outside layer can be configured to form a ring. The adhesive system can include an inside layer, wherein the inside layer is composed of a material with a high moisture vapor transmission rate. The inside layer can be joined with the outside layer such that a small gap is formed between the inside layer and the outside layer.

Disclosed is an adhesive system for adhering a medical device to the skin of a patient. In some examples, the adhesive system can include an outside layer and an inside layer. In some embodiments, the outside layer is elastic and re-sealable to the skin of the patient. In other embodiments, the outside layer is configured to form a ring. In some embodiments, the inside layer is composed of a material with a high moisture vapor transmission rate. In other embodiments, the inside layer can be joined with the outside layer such that a small gap is formed between the inside layer and the outside layer.

Additional embodiments of the adhesive system are directed to multilayer adhesive systems. In some embodiments, the multilayer adhesive system comprises a first layer that includes a first layer adhesive for attaching to skin. The first layer: (i) has a first area defined by a first perimeter, (ii) has a first layer effective elastic modulus that is maintained for a first strain, and (iii) comprises a material having a first layer inherent elastic modulus that is higher than the first layer effective elastic modulus. The adhesive system also includes a second layer that is attached to the first layer. The second layer: (i) has a second area defined by a second perimeter, where portions of the second area extend beyond the first perimeter, (ii) provides mechanical reinforcement to the first layer, (iii) has a second layer effective elastic modulus that is maintained for a second strain, and (iv) comprises a material having a second layer inherent elastic modulus that is higher than the second layer effective elastic modulus. In some embodiments, the multilayer adhesive system has an effective system elastic modulus that is maintained for a third strain.

Multilayer adhesive system embodiments disclosed herein can comprise a first layer that includes a first layer adhesive for attaching to skin, where the first layer: (i) has a first area defined by a first perimeter and (ii) comprises a material having a first layer inherent elastic modulus. The system also includes a second layer attached to the first layer, where the second layer: (i) has a second area defined by a second perimeter, wherein portions of the second area extend beyond the first perimeter, (ii) provides mechanical reinforcement to the first layer, (iii) has a second layer effective elastic modulus that is maintained for a second strain, and (iv) comprises a material having an second layer inherent elastic modulus that is higher than the second layer effective elastic modulus. In some embodiments, the multilayer adhesive system has an effective system elastic modulus that is maintained for a third strain.

Certain embodiments are directed to adhesive systems that include a first layer that comprises a first material that has a first inherent elastic modulus, a plurality of first layer perforations that form a plurality of discontinuous portions in the first layer, a first area defined by a first perimeter, an adhesive for attaching to skin, and a first effective elastic modulus that is lower than the first inherent elastic modulus. The adhesive system also includes a second layer that comprises a second material that has a second inherent elastic modulus, a plurality of second layer perforations, a second area defined by a second perimeter, an adhesive for attaching to the first layer, and a second effective elastic modulus that is lower than the second inherent elastic modulus.

The adhesive systems disclosed herein also include embodiments directed composite adhesive system that comprise a first layer for attaching to skin, where the first layer has (i) a first area defined by a first perimeter and (ii) a first layer inherent elastic modulus that is maintained for a first strain. The system also includes a second layer that is attached to the first layer, where the second layer (i) has a second area substantially equal to the first area and a second perimeter substantially equal to the first perimeter, (ii) provides mechanical reinforcement to the first layer, (iii) has a second layer effective elastic modulus that is maintained for a second strain, and (iv) comprises a material having a second layer inherent elastic modulus that is higher than the second layer effective elastic modulus. Lastly, the system includes a third layer that is attached to the second layer with a third layer adhesive. The third layer: (i) has a third area defined by a third perimeter, (ii) provides mechanical reinforcement to the second layer, (iii) has a third layer effective elastic modulus that is maintained for a third strain, and (iv) comprises a material having a third layer inherent elastic modulus that is higher than the third layer effective elastic modulus. In some embodiments, the composite adhesive system has an adhesive system effective elastic modulus that is maintained for a fourth strain.

Additional embodiments of a three-layer adhesive system include a first layer that has a first layer adhesive for attaching to skin, where the first layer: (i) has a first area defined by a first perimeter, (ii) has a first layer effective elastic modulus that is maintained for a first strain, and (iii) comprises a material having a first layer inherent elastic modulus that is higher than the first layer effective elastic modulus. The second layer, which is attached to the first layer, (i) has a second area substantially equal to the first area and a second perimeter substantially equal to the first perimeter, (ii) provides mechanical reinforcement to the first layer, (iii) has a second layer effective elastic modulus that is maintained for a second strain, and (iv) comprises a material having a second layer inherent elastic modulus that is higher than the second layer effective elastic modulus. The third layer is attached to the second layer with a third layer adhesive. The third layer: (i) has a third area defined by a third perimeter, (ii) provides mechanical reinforcement to the second layer, (iii) has a third layer effective elastic modulus that is maintained for a third strain, and (iv) comprises a material having a third layer inherent elastic modulus that is higher than the third layer effective elastic modulus. In some embodiments, the multilayer adhesive system has an adhesive system effective elastic modulus that is maintained for a fourth strain.

Embodiments are also directed to a method of wearing an adhesive system where the method comprises the steps of providing an adhesive system that includes a first layer made a first material with a first inherent elastic modulus and having, a plurality of first layer perforations that form a plurality of discontinuous portions in the first layer, a first area defined by a first perimeter, an adhesive for attaching to skin, and a first effective elastic modulus that is lower than the first inherent elastic modulus. The system also includes a second layer made of a second material having a second inherent elastic modulus and having a plurality of second layer perforations, a second area defined by a second perimeter, an adhesive for attaching to the first layer, and a second effective elastic modulus that is lower than the second inherent elastic modulus. The method includes applying the adhesive system to skin, applying a tensile force to the adhesive system to achieve a strain of up to 0.4, causing at least one discontinuous portion in the first layer to separate from an adjacent discontinuous portion in the first layer, forming concentrated areas of stress between adjacent second layer perforations, causing the second layer to plastically deform under the applied tensile force, and removing the tensile force.

Further embodiments of the invention are directed to an adhesive system, having a first layer including a first layer material with a top and a bottom having a bottom perimeter, and a first layer adhesive on the bottom for attaching to skin, the first layer having an inherent modulus of elasticity. The adhesive system also includes a second adhesive that is more aggressive than the first layer adhesive, the second adhesive along only the bottom perimeter. The first layer includes a plurality of modifications therein that result in the first layer having an effective modulus of elasticity that is lower than the inherent modulus of elasticity of the first layer. In some embodiments, the plurality of modifications in the first layer are a plurality of perforations.

Additional embodiments of the invention are directed to an adhesive system that includes a circular spun lace non-woven material that comprises a top and a bottom having a bottom perimeter, and an adhesive on the bottom for attaching to skin where the spun lace non-woven material has an inherent modulus of elasticity. The adhesive system also includes a hydrocolloid material ring around only the bottom perimeter of the spun lace non-woven material. In this embodiment, the spun lace non-woven material includes a plurality of rings having a plurality of perforations that transform the adhesive system into an adhesive system having an effective modulus of elasticity that is lower than the inherent modulus of elasticity of the spun lace non-woven material.

In some embodiments, the invention is directed to an adhesive system having at least one layer comprising a material having a top and a bottom including a bottom perimeter, a first adhesive on the bottom for at least partially attaching to skin, a first inherent modulus of elasticity, and a plurality of modifications therein that result in the first layer having an effective modulus of elasticity that is lower than the first inherent modulus of elasticity. In these embodiments, the adhesive system also includes a second adhesive that is more aggressive than the first adhesive, the second adhesive disposed along only the bottom perimeter.

Furthermore, embodiments of the invention are directed to an adhesive system that includes a top layer having a top layer material and a top layer adhesive, a bottom layer having a bottom layer material with a first side and a second side and a bottom layer adhesive on the second side, a plurality of layers between the top layer and the bottom layer, each of the plurality of layers having a layer material and a layer adhesive, and an aggressive adhesive that is more aggressive than the bottom layer adhesive, the aggressive adhesive disposed upon only a portion of the second side of the bottom layer, the aggressive adhesive for attaching to skin. In these embodiments, at least one of the top layer, the bottom layer and the plurality of layers includes a plurality of modifications therein that result in said layer having an effective modulus of elasticity that is lower than the inherent modulus of elasticity of said layer. In some embodiments, the plurality of modifications are a plurality of perforations.

In some embodiments the invention is directed to a medical device that comprises an analyte sensor and an adhesive system. Embodiments of the adhesive comprise a first layer including a first layer material having a top and a bottom having a bottom perimeter and a first layer adhesive on the bottom for attaching to skin where the first layer has an inherent modulus of elasticity, and a second adhesive that is more aggressive than the first layer adhesive where the second adhesive is included along only the bottom perimeter. In some embodiments, the first layer includes a plurality of modifications therein that result in the first layer having an effective modulus of elasticity that is lower than the inherent modulus of elasticity of the first layer. In some embodiments, the analyte sensor senses an analyte selected from the group comprising glucose, galactose, fructose, lactose, peroxide, cholesterol, amino acids, alcohol, lactic acid, and mixtures of the foregoing. In some embodiments, the plurality of modifications are a plurality of perforations.

Additional embodiments of the invention are directed to a medical device that comprises an analyte an analyte sensor and an adhesive system. In some embodiments, the adhesive system comprises at least one layer having a material with a top and a bottom having a bottom perimeter, a first adhesive on the bottom for at least partially attaching to skin, a first inherent modulus of elasticity, a plurality of modifications therein that result in the first layer having an effective modulus of elasticity that is lower than the first inherent modulus of elasticity, and a second adhesive that is more aggressive than the first adhesive, the second adhesive along only the bottom perimeter. In some embodiments, the plurality of modifications are a plurality of perforations. In some embodiments, the analyte sensor senses an analyte selected from the group comprising glucose, galactose, fructose, lactose, peroxide, cholesterol, amino acids, alcohol, lactic acid, and mixtures of the foregoing.

In some embodiments the invention is directed to an apparatus that comprises a medical device and an adhesive system. Embodiments of the adhesive comprise a first layer including a first layer material having a top and a bottom having a bottom perimeter and a first layer adhesive on the bottom for attaching to skin where the first layer has an inherent modulus of elasticity, and a second adhesive that is more aggressive than the first layer adhesive where the second adhesive is included along only the bottom perimeter. In some embodiments, the first layer includes a plurality of modifications therein that result in the first layer having an effective modulus of elasticity that is lower than the inherent modulus of elasticity of the first layer. In some embodiments, the medical device is a body wearable medical device. In some embodiments, the plurality of modifications are a plurality of perforations.

Additional embodiments of the invention are directed to an apparatus that comprises a medical device and an adhesive system. In some embodiments, the adhesive system comprises at least one layer having a material with a top and a bottom having a bottom perimeter, a first adhesive on the bottom for at least partially attaching to skin, a first inherent modulus of elasticity, a plurality of modifications therein that result in the first layer having an effective modulus of elasticity that is lower than the first inherent modulus of elasticity, and a second adhesive that is more aggressive than the first adhesive, the second adhesive along only the bottom perimeter. In some embodiments, the plurality of modifications are a plurality of perforations. In some embodiments, the medical device is a body wearable medical device such as, for example, a pump for the delivery of therapeutic drugs.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting. Throughout the drawings, similar symbols typically identify similar components, unless context dictates otherwise. Note that the relative dimensions of the following Figures may not be drawn to scale.

FIGS. 1A-C are an exploded, side, and top view of the adhesive system for attaching an opto-enzymatic device to the surface of skin, according to an embodiment of the present invention.

FIG. 2A is a top view of an adhesive system for attaching an opto-enzymatic device to the surface of skin, according to an embodiment of the present invention.

FIG. 2B is a cross-sectional view taken along line A-A in FIG. 2A. FIG.

FIG. 2C is a top view of an adhesive system on skin in a relaxed state, according to an embodiment of the present invention.

FIG. 2D is a top view of the adhesive system depicted in FIG. 2C on a skin when the skin is stretched, according to an embodiment of the present invention.

FIG. 2E is a top view of an adhesive system, according to an embodiment of the present invention.

FIG. 2F is an exploded view of the adhesive system in FIG. 2E, according to an embodiment of the invention.

FIG. 2G is top view of the top layer of the adhesive system in FIG. 2E, according to an embodiment of the invention.

FIG. 2H is a front perspective view of the bottom layer of the adhesive system in FIG. 2E, according to an embodiment of the invention.

FIG. 2I is a detail of the perforations in the top layer of the adhesive system in FIG. 2G, according to an embodiment of the invention.

FIG. 2J is a bottom view of the adhesive system in FIG. 2E, according to an embodiment of the invention.

FIG. 2K is an exploded view of an adhesive system, according to an embodiment of the invention.

FIG. 2L is an exploded view of an adhesive system, according to an embodiment of the invention.

FIG. 2M is a top view of an adhesive system, according to an embodiment of the present invention.

FIG. 2N is an exploded view of the adhesive system in FIG. 2M, according to an embodiment of the invention.

FIG. 2O is an exploded view of an adhesive system, according to an embodiment of the invention.

FIG. 2P is a bottom view of the adhesive system in FIG. 2O, according to an embodiment of the invention.

FIG. 2Q is an exploded view of an adhesive system, according to an embodiment of the invention.

FIG. 2R is a bottom view of the adhesive system in FIG. 2Q, according to an embodiment of the invention.

FIG. 2S is a detail of the modifications to the adhesive system layers, according to an embodiment of the present invention.

FIG. 2T is a chart summarizing strain test results for different adhesive system embodiments according to the present invention.

FIG. 2U is an illustration of an adhesive system, according to an embodiment of the invention, attached to relaxed skin.

FIG. 2V is an illustration of the adhesive system depicted in FIG. 2U on skin when the skin is in a stretched state.

FIG. 2W is an illustration of the adhesive system depicted in FIG. 2V on skin when the skin returned to a relaxed state.

FIG. 3A is a top view of an adhesive system, according to an embodiment of the invention.

FIG. 3B is a cross-sectional view taken along line A-A in FIG. 3A.

FIG. 3C is a bottom view of the adhesive system depicted in FIG. 3A.

FIG. 3D is a top view of an adhesive system, according to an embodiment of the invention.

FIG. 3E depicts a plurality of perforations in an unstrained state, according to an embodiment of the invention.

FIG. 3F depicts the plurality of perforations shown in FIG. 3E in a strained state, according to an embodiment of the invention.

FIG. 4A is a top view of an adhesive system, according to an embodiment of the invention.

FIG. 4B is a chart summarizing strain test results of modified and unmodified adhesive systems, according to embodiments of the present invention.

FIG. 5 depicts an adhesive system, according to an embodiment of the invention.

FIG. 6A is a schematic view of the flow of moisture from the surface of the skin through an adhesive system and attached opto-enzymatic sensor system, according to an embodiment of the present invention.

FIG. 6B is a schematic view of the flow of moisture from the surface of the skin through an adhesive system and attached opto-enzymatic sensor system, according to an embodiment of the present invention.

### DETAILED DESCRIPTION

The disclosed and described technology relates to single layer and multilayer adhesive systems. The adhesive systems described herein include at least one layer of material having some type of modification therein that reduces the modulus of elasticity of that material. Additionally, the adhesive systems can include a ring of an aggressive adhesive such as, for example, a hydrocolloid, around its bottom perimeter that is to contact skin.

Disclosed herein are embodiments of a multilayer composite adhesive system, configured to adhere, in some embodiments, to an body wearable device, such as, for example, opto-enzymatic analyte sensors that can be used to measure, for example, glucose, galactose, fructose, lactose, peroxide, cholesterol, amino acids, alcohol, lactic acid, and mixtures of the foregoing, to the surface of skin. The multilayer composite adhesive systems disclosed herein can attach to the bottom of the body wearable device housing thereby allowing the device to be attached to the skin for an extended period of time, for example, 4 to 7 days, 7 to 10 days, 10 to 14 days or 14 to 21 days.

Embodiments of the adhesive systems disclosed and described herein can be used to attach any medical device or medical appliance to the skin. Example medical devices and medical appliances include, and are not limited to, analyte sensors, pumps for the delivery of therapeutic drugs (insulin, chemotherapy drugs, etc.), and any other body wearable medical device or appliance as will be readily understood by those of skill in the art. In some embodiments, the adhesive systems disclosed and described herein can be used for wound care. Example wound care uses include, and are not limited to, tapes to attach wound dressings to skin, bandages, and any other wound care use as will be readily understood by those of skill in the art.

In order to achieve the required sustained attachment to the skin while allowing the adhesive system have a high moisture vapor transmission rate (“MVTR”) and to be easily removed from the skin when desired, embodiments of the present invention are directed to multilayer composite adhesive systems where the properties of the layers combine to form a system with a high MVTR that addresses the mismatch/differences in the mechanical properties between the skin and the adhesive system, i.e., stress/strain differentials that exist between skin and the adhesive systems and that uses a skin adhesive that provides sufficient adhesion to skin while allowing the adhesive system to be easily removed with little pain. Thus, each layer of the present adhesive systems can have different mechanical and material properties but when the properties of all layers are combined, they address the issues with prior systems by mimicking skin mechanics in order to address the strain mismatch between the skin and the adhesive system while providing a high MVTR.

To satisfy these requirements, the multilayer composite adhesive systems of the embodiments of the present invention have been designed to have a high MVTR and a low, effective Young's/elastic modulus. Further, the system can plastically deform when worn on the skin and has good adhesion to skin while being easily removed from the skin when desired. The MVTR of a material can be an inherent property of the material or a material's MVTR can be changed/adjusted by altering the material to include, for example, openings, slits, cuts or other perforations (collectively, “perforations”) therein, resulting in a material that has a higher effective MVTR, thereby providing a pathway for moisture to escape through the material. As used herein, (1) “inherent” shall mean a property of an unmodified material or layer or multilayer material and (2) “effective” shall mean the resulting property after a material or layer or multilayer adhesive system has been modified, for example, as disclosed herein to include modifications such as perforations or the resulting properties of a multilayer adhesive system constructed in accordance with the embodiments disclosed herein.

A material typically plastically deforms when its linear elastic force is exceeded as stress is developed in the material. Similar to a material's MVTR, a material's elastic modulus can be an inherent property of the material or it can be changed/adjusted by modifying the material to include, for example, perforations therein, resulting in a material that has an effective elastic modulus that is lower than its inherent elastic modulus. The shape, orientation, size and spacing of these perforations, can also be used to change a material's elastic in different directions, i.e., the web and cross-web directions of the material, depending on the size, orientation and spacing of the perforations.

For example, as discussed in detail below, a material that includes perforations that are longer in length than the gap/spacing between adjacent perforations will have a lower effective elastic modulus than a material that includes perforations that are shorter in length than the gap/spacing between adjacent perforations. Using perforations that have different lengths and spacing between in different directions allows tuning of the modulus of elasticity in the different directions, i.e. a first modulus of elasticity in a first direction and a second modulus of elasticity in a second direction where the first and second elastic modulus's can be the same or different. As discussed in more detail below, the length of the perforations and the spacing between adjacent perforations can be adjusted to tune the effective elastic modulus of the materials/layers and hence, the effective modulus of the embodiments of the adhesive systems disclosed and described herein. For example, the effective elastic modulus of an individual layer or the constructed multilayer adhesive system can be tuned/adjusted to be less than approximately 100 Kpa, 90 Kpa, 70 Kpa, 60 Kpa, 50 Kpa, 40 Kpa, 30 Kpa, 20 Kpa, and 10 Kpa, at 100% strain.

Thus, embodiments of the present adhesive systems have been designed to have a high MVTR and low elastic modulus, i.e., designed to have low elasticity, that undergo plastic deformation at low strains. Having an adhesive system that plastically deforms when attached skin, allows the system to use a less aggressive adhesive to attach the adhesive system to the skin as the shear forces between the adhesive and the skin are significantly reduced after the adhesive system plastically deforms. Adhesive systems that plastically deform when worn on the skin, solves the issue of edge peel and results in an adhesive system that remains attached to the skin for an extended period of time, for example, five (5) weeks.

The multilayer, composite adhesive system embodiments disclosed herein are also advantageous as they permit different system designs based on the intended use of the system while allowing one to design the system to have the required MVTR and elastic modulus properties. For example, one may desire to have an adhesive system with moisture wicking properties, or one may desire to have an adhesive system to absorb bodily fluid such as in the form of a bandage, or one may desire to an adhesive system with sufficient strength to attach medical devices and other medical items to the body. Different uses may require different properties or a combination of properties, which can be achieved through the use of layers of different materials, which individually may not meet the intended use requirements but when modified as discussed herein and combined, provide the required properties.

Material properties to consider in designing adhesive system embodiments of the present invention include, and are not limited to, Young's modulus, MVTR, hydrophobicity, hydrophilicity and moisture wicking, adhesive strength, adhesive hypoalgernicity and intact adhesive system removal.

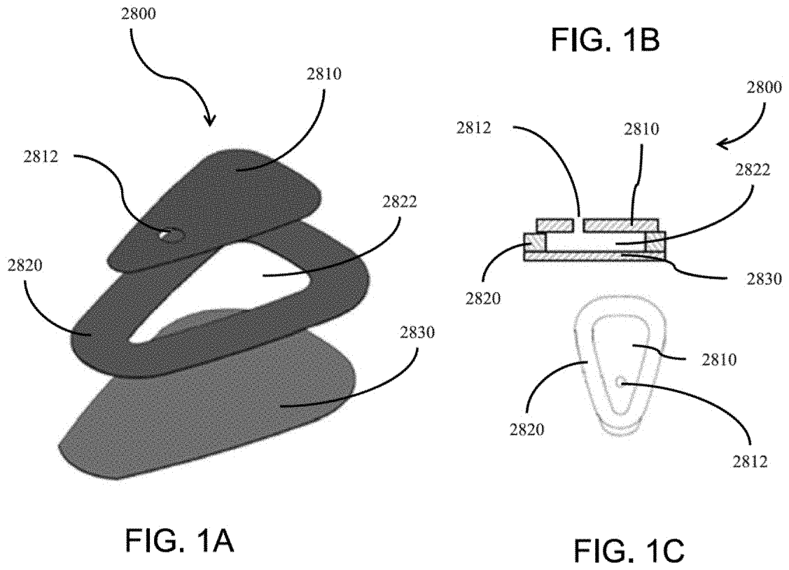

FIGS. 1A-C illustrates exploded and side views of an embodiment of the adhesive system 2800. The adhesive system 2800 is a multilayer adhesive system that provides a high MVTR in general, especially under the housing of the attached device. In some examples, the adhesive system 2800 includes a first layer composed of a device adhesive 2830, a second layer composed of the outer ring 2820, and a top layer composed of the coin standard 2810. The adhesive system 2800 can be oriented such that the first layer device adhesive 2830 is attached to the bottom of the device and the third layer coin standard 2810 is attached to the surface of the skin.

Turning first to the coin standard 2810, in some examples the coin standard 2810 is attached to the skin. The surface of the coin standard 2810 can be composed of an acrylate pressure sensitive adhesive on a PET release. The pressure sensitive adhesive allows the coin standard 2810 to adhere to the skin when pressure is applied—thereby activating the adhesive without the use of a solvent, water or heat. The material of the coin standard 2810 can be composed of a spun lace non-woven material with a high MVTR. In some examples, the coin standard 2810 can have a thickness of 4 mm.

As illustrated in FIGS. 1A to 1C, the coin standard 2810 can include an opening 2812 that extends through the coin standard 2810. In some examples, the opening 2812 can have a diameter of 3 mm and can be placed a distance of 10 mm from the narrow end of the coin standard 2810.

Turning next to the outer ring 2820, in some examples, the outer ring 2820 is composed of a re-attachable pressure sensitive adhesive. The outer ring 2820 can be composed of a lined silicon/silicon pressure sensitive adhesive on a PTFE release.

In some examples, the outer ring 2820 can be joined to the coin standard 2810. The attachment between the two layers can form a gap 2822. The outer ring 2820 can be attached to the coin standard 2810 with acrylate pressure sensitive adhesive. In some examples, the acrylate pressure sensitive adhesive can be a polyurethane acrylate (P-UR acrylate). In some embodiments, the release liner of the outer ring 2820 is formed from a patterned PET and PTFE pattern. The PET can be bonded to the PTFE below the coin and the PTFE below the silicon. In some examples, the outer ring 2820 can have a base width of 30 mm and a length of 40 mm. In some embodiments, outer ring 2820 can have a width of between approximately 3 mm and 10 mm and a thickness between approximately 0.025 mm and 0.1 mm.

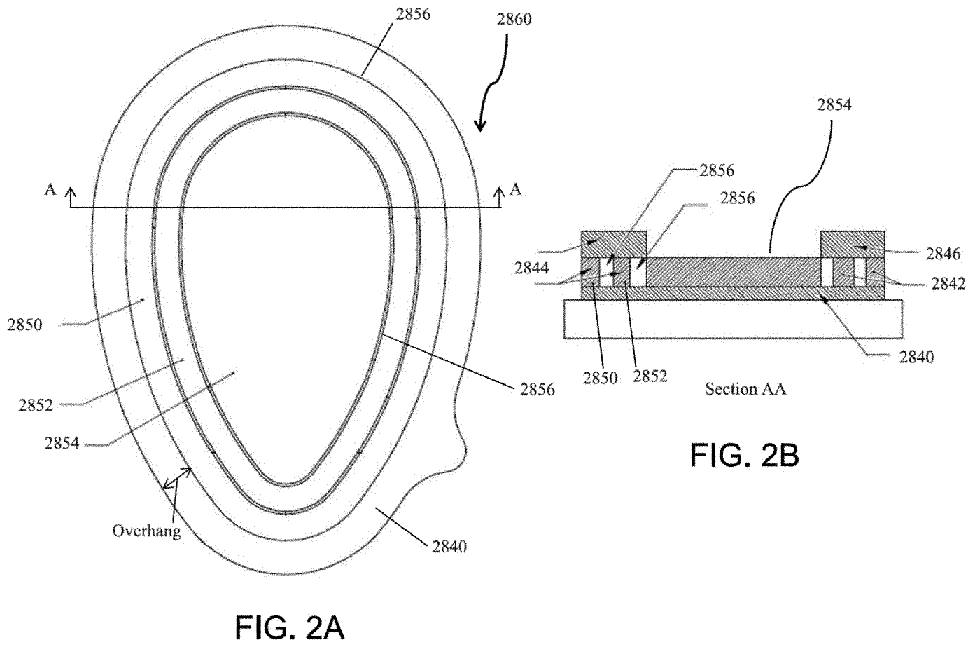

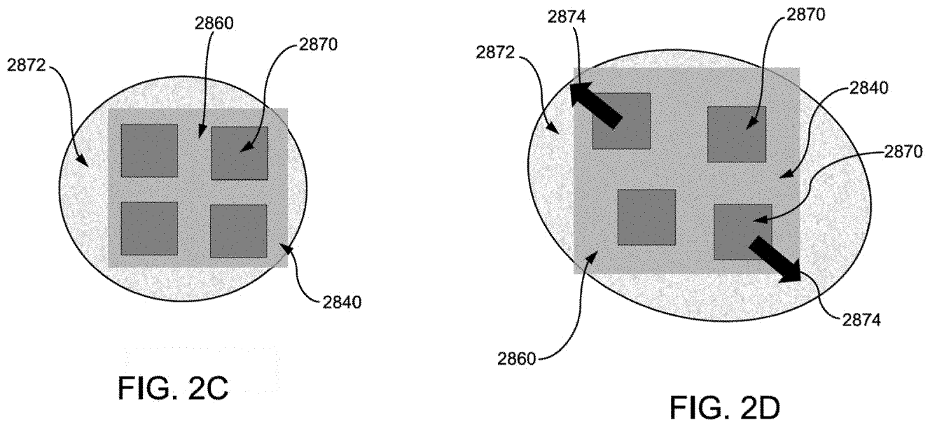

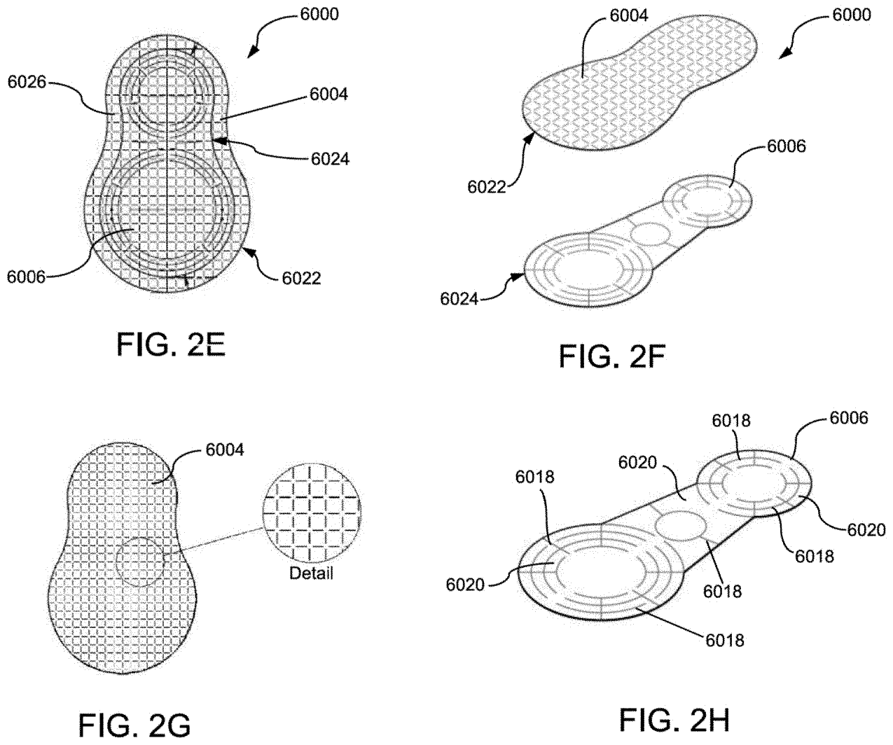

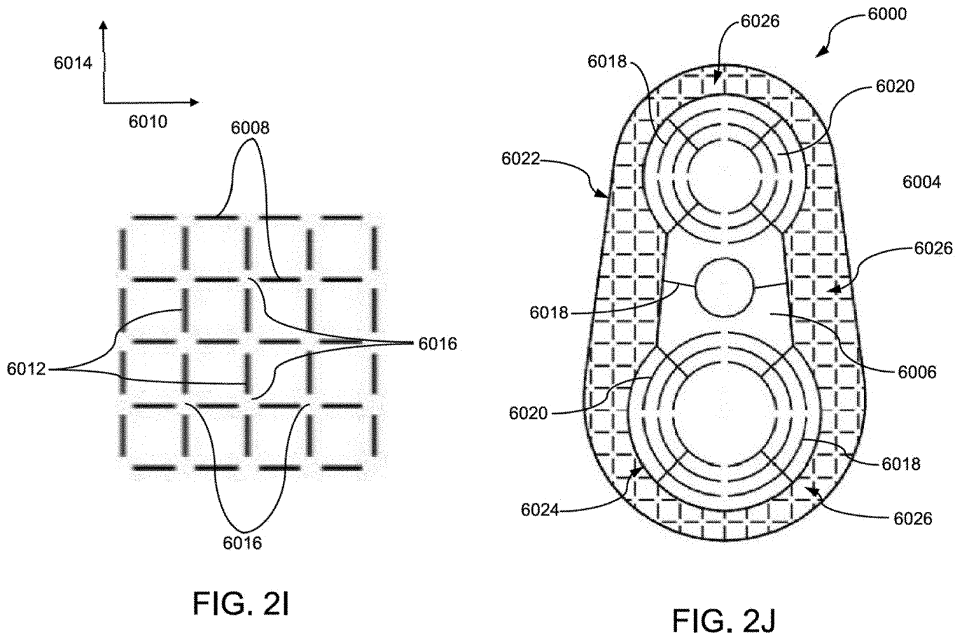

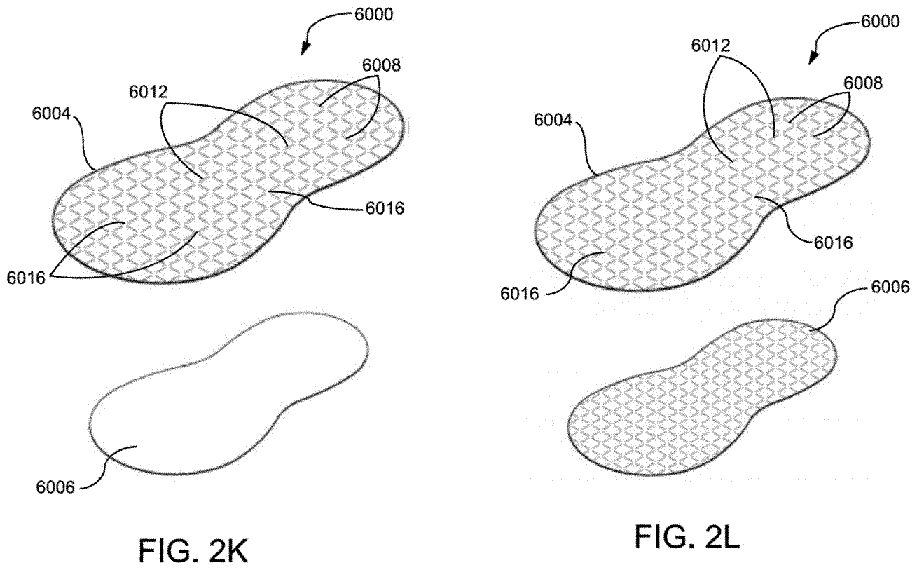

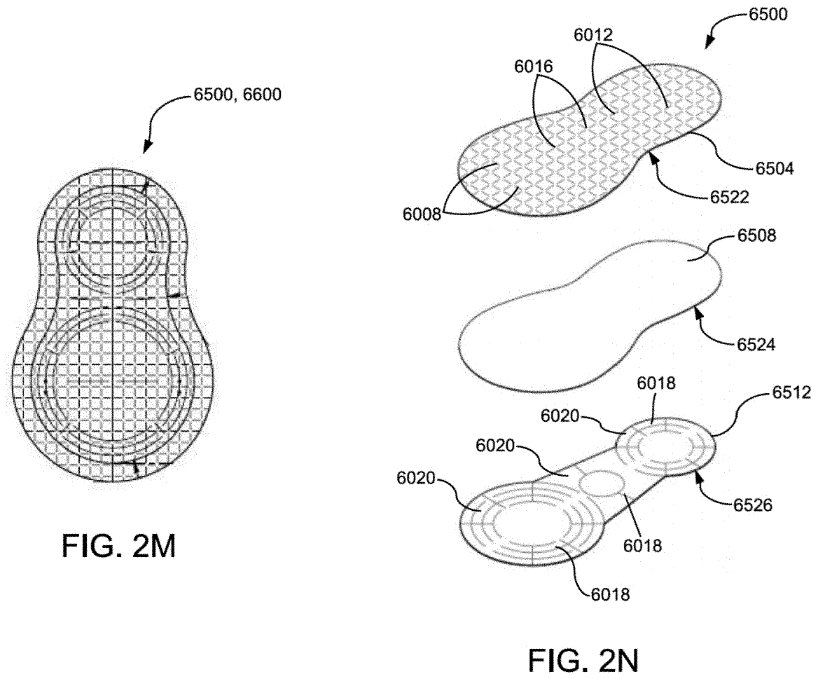

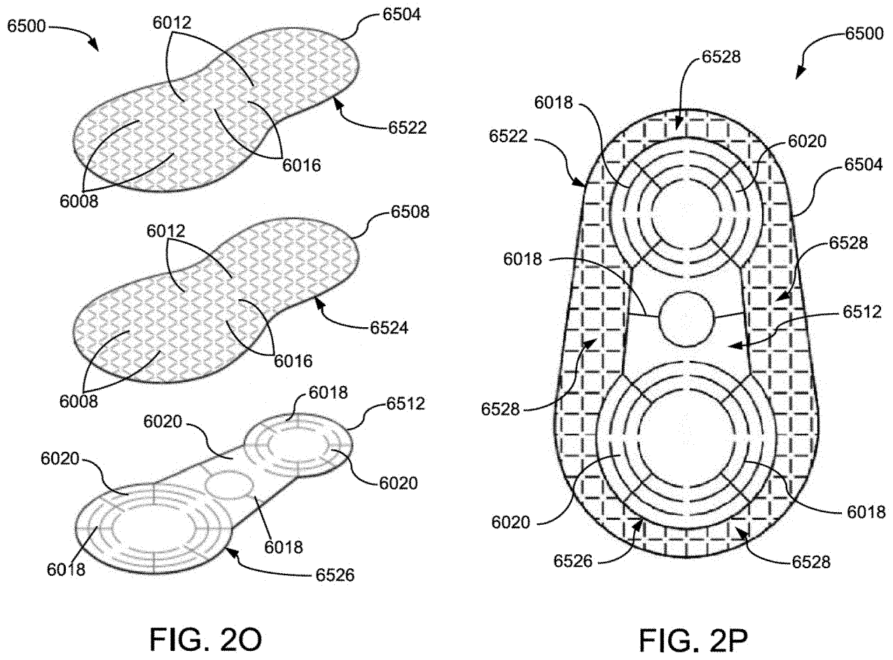

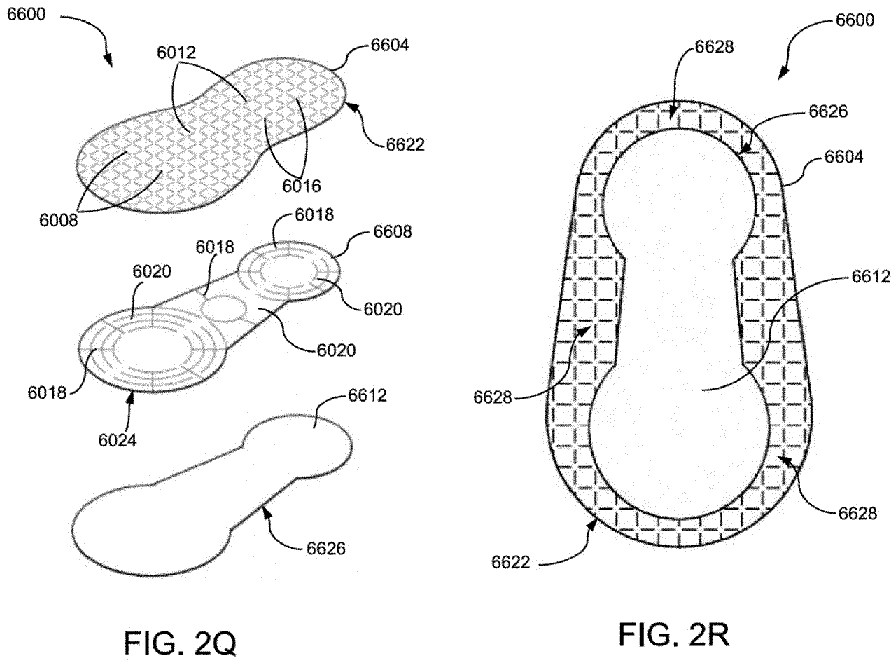

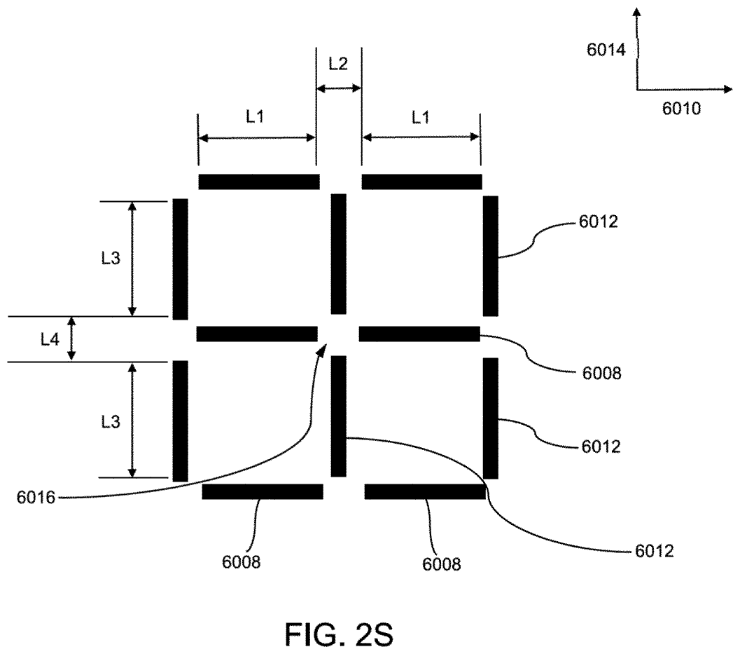

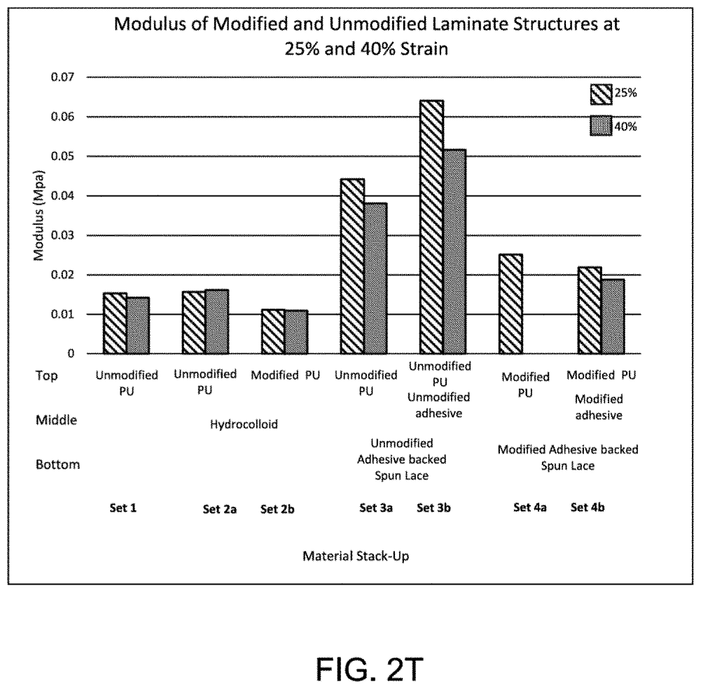

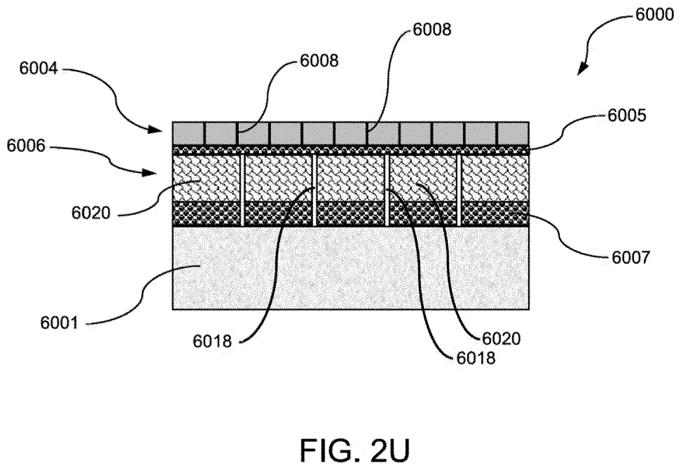

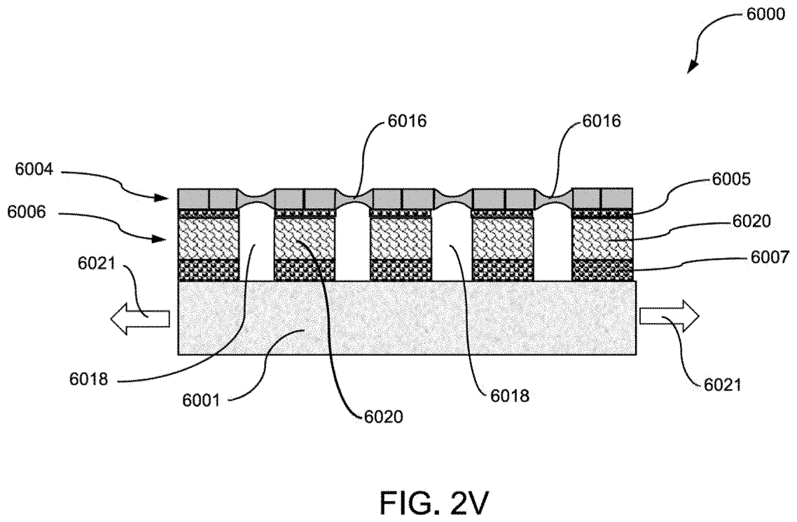

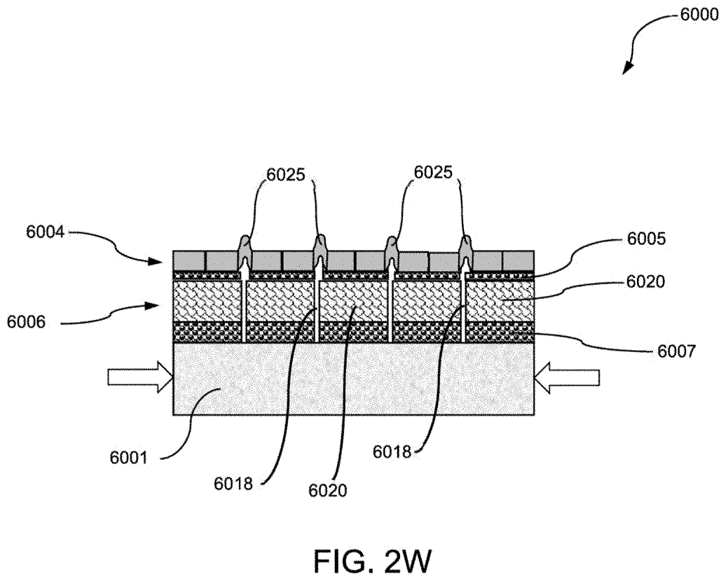

FIGS. 2A-B illustrate a top and side view of another embodiment of the adhesive system 2860. The adhesive system 2860 illustrated in FIGS. 2A-B is a multi-layered system that includes a top layer 2840 with a top layer adhesive 2842 and a bottom layer 2844 with a bottom layer adhesive 2846. The top layer 2840 can be formed from a material having a low inherent elastic modulus or it can be made from a material that has been modified (as discussed in more detail below) to have a low effective elastic modulus. Example materials for the top layer include polyurethane and a silicone elastomer. The bottom layer 2844 includes an outer ring 2850, a middle ring 2852, a central portion layer 2854, and gaps 2856, which can be continuous or discontinuous. The outer ring 2850 can include a number of variations. In some examples, the outer ring 2850 is a high strength bio-compliant skin adhesive that can be connected to the top layer 2840 of the adhesive system 2860. The bottom layer 2844 can include a middle ring 2854 and a central portion 2854 of spun lace, non-woven material, which can be a material that wicks moisture, such as perspiration, away from under the device.

In other examples, the bottom layer 2844 can be a spun lace, non-woven material that includes a plurality of cuts or gaps 2856 therein that divide the bottom layer 2844 into an outer ring 2850, a middle ring 2852 and a central portion 2854. In this embodiment, the bottom layer adhesive 2844 can be more aggressive than the top layer adhesive 2842.

In another embodiment, the outer annular region 2850 can be a re-attachable bio-compliant skin adhesive connected to the top layer 2840 of the adhesive system 2860. The outer annular region 2850 can have a central portion 2854 of spun lace, non-woven material. The outer annular region 2850 may also have an additional adhesive layer above the central portion 2854 of spun lace, non-woven material. In other examples, the outer annular region 2850 can have the same materials as the central portion 2854. As well, the outer annular region 2850 can have an adhesive connected to the top layer 2840 of the adhesive system 2860.

In some examples, the adhesive system 2860 includes a top layer 2850 that can be a backing material that has a high MVTR, such as polyurethane. In some examples, the backing material is thin and complaint. In some embodiments, as illustrated in FIG. 2B, one or more layers can include one or more physical gaps 2856. In some examples, these gaps 2856 can be in the spun lace, non-woven material of the bottom layer 2844 and adhesive layer below the backing of the top layer 2852 creating discontinuous segments. The physical gaps 2856 provide strain relief in the adhesive system 2860 as the adhesive system 2860 is stretched, allowing the discontinuous segments of the annular region to move independently of one another. In some examples, additional gaps through the entire adhesive system 2860 can provide further strain relief. In some examples, these additional gaps in the spun lace and skin adhesive can provide further strain relief. While in the figures, these gaps 2854 are shown as extending completely through the material, it should be noted that these gaps can also be recessed, indented or embossed portions of the material, which create failure lines in the material that are designed to fail and hence, cause gaps to form in the material, when stress is applied to the material, thereby providing the required strain relief.

In another embodiment of the adhesive system 2860 depicted in FIGS. 2 C and 2D, instead of the bottom layer being divided into ring-shaped discontinuous portions, the bottom layer 2844 can be divided into polygonal-shaped discontinuous portions 2870. The top layer 2840 can be formed from a material having a low inherent elastic modulus or it can be made from a material that has been modified (as discussed in more detail below) to have a low effective elastic modulus. The top layer 2840 may be attached to the bottom layer 2844 with an adhesive. The bottom layer 2844 can be a spun lace, non-woven material that includes an adhesive for attaching to the skin 2872. FIG. 2C depicts the adhesive system 2860 adhered to skin 2872 when the skin is in a relaxed state. When adhered to the skin 2872, the discontinuous portions 2870 form discrete attachment points to the skin 2872. As depicted in FIG. 2D, when the skin 2872 is stressed/stretched as indicated by arrows 2874, because the top layer 2840 has a low elastic modulus either inherently or through modification as discussed herein, the discontinuous portions 2870 that are adhered to the skin 2872 easily move with the skin in the direction of arrows 2874. The combination of the bottom layer 2844 having discrete attachment points between the discontinuous portions 2870 and the skin 2872 and the top layer 2840 having a low elastic modulus that stretches and/or plastically deforms under stress, provides the required strain relief between the skin 2872 and the adhesive system 2860.

In the herein disclosed embodiments, dividing the bottom layer of the adhesive system into multiple annular regions or other discontinuous portions, helps to minimize the strain on the inner or central regions of the adhesive system by distributing stress across the annular regions or discontinuous portions. Adhesive systems constructed in this manner, create a stress-strain gradient between the inner or central regions and the ring or discontinuous portions that extend away from the inner or central regions. For example, the embodiment of the adhesive system depicted in FIGS. 2A and 2B includes a bottom layer 2844 with discontinuous portions (annular regions 2850, 2852) that are detached from a central portion (central portion 2854). In this embodiment, a device, such as an opto-enzymatic device as disclosed herein, may be included on the adhesive system in the area above central portion 2854 (a loaded portion). Thus, designing an adhesive system that has a central loaded portion with discontinuous portions extending away from the central loaded portion (see for example, FIGS. 2C and 2D), allows for the stresses on the loaded central portion to be distributed across the exterior discontinuous portions.

In some examples, the adhesive system 2800 is re-sealable and provides for comfortable adhesion. The illustrated adhesive system 2800 can include two zones of attached materials. In some embodiments, the outer layer can be elastic, with a low durometry. The outer layer can allow the adhesive system 2800 and attached device to be re-sealable to the skin. In some embodiments, the inner layer can be composed of a material that is less elastic but has a high MVTR. As will be discussed in further detail below, the material properties of the inner layer can allow the skin to breath by allowing water and/or water vapor to evaporate off the surface of the skin.

Depicted in FIGS. 2E to 2J is another embodiment of the present adhesive system. The adhesive system 6000 is a two-layer system that includes a top layer 6004 and a bottom layer 6006. The top layer 6004 can be made from a material having an inherent low elastic modulus and an inherent high MVTR or it can be made from a material that is modified to have an effective lower elastic modulus and/or an effective higher MVTR. The top layer 6004 can include an adhesive for attaching the top layer 6004 to the bottom layer 6006. Thus, a material having a higher elastic modulus and/or a lower MVTR than desired may be used but may be modified mechanically, for example, to include a plurality of modifications, such as, for example, perforations 6008, along a first direction 6010, and/or a plurality of modifications, such as, for example, perforations 6012, along a second direction 6014 (as depicted in FIGS. 2G and 21, that extend through the thickness of the top layer 6004 and which can also extend through the adhesive.

The plurality of perforations 6008, 6012 transform the top layer material from a material having a high or first inherent elastic modulus and/or a low inherent MVTR into a material having an effective lower or second elastic modulus and/or an effective higher MVTR. The effective low elastic modulus is achieved by creating stress relaxing perforations that expand as the material is stretched. As the perforations expand, a plurality of concentrated areas of stress 6016 develop between adjacent perforations 6008, 6010, that undergo plastic deformation when stress is applied to the top layer 6004. Because any stress that is applied to the top layer 6004 is concentrated in areas 6016, these concentrated areas of stress 6016 plastically deform under external loads that are lower than stress that would cause an unmodified top layer 6004 material to plastically deform. This plastic deformation provides further strain relief between the top layer 6004 and the skin. The stress becomes lower for a given strain after deformation. Although the perforations 6008, 6012 in this embodiment are shown in a cross-hatch orthogonal pattern, the perforations 6008, 6012 can have any shape or pattern as long as they allow the material to separate creating a low elastic modulus response and preferentially create concentrated areas of stress 6016 between adjacent perforations. Additionally, in some embodiments, the plurality of perforations 6008, 6012 may extend completely through the top layer 6004 material while in other embodiments, they may not extend completely through the thickness of the material/layer and instead may be recessed, indented or embossed portions that fail when under stress and create the concentrated areas of stress 6016 between adjacent indentations causing the material layer to plastically deform under stress when applied to skin. In some embodiments, the top layer 6004 is a polyurethane material. In some embodiments, the top layer is a silicone elastomer.

The bottom layer 6006 can comprise any material (wicking materials, adhesives, etc.) and the material should be chosen based on the intended use of the adhesive system. In some embodiments, the material for the bottom layer 6006 is a wicking material such as, for example, a spun lace non-woven material, that includes an adhesive for adhering the bottom layer 6006 to skin. The wicking material of the bottom layer 6006, which contacts the skin, transports moisture laterally from areas of high moisture to areas of low moisture. As illustrated in FIGS. 2E, 2F, 2H and 2J, the bottom layer 6006 includes a plurality of perforations 6018 therein that form a plurality of discontinuous portions 6020. These perforations 6018 can be continuous or discontinuous. Accordingly, when the bottom layer 6006 is adhered to skin and is stressed, the plurality of discontinuous portions 6020 separate from each other, thereby providing strain relief in the bottom layer 6006. Because the discontinuous portions 6020 are adhered to the skin, as they separate and move away from the adjacent discontinuous portions 6020, they move with the skin, independently of one another. Although, in some embodiments, the plurality of perforations 6018 may extend completely through the bottom layer 6006 material, they may also be recessed, indented or embossed portions of the material, which create failure lines in the material that are designed to fail under stress and hence, cause adjacent discontinuous portions 6020 to separate from one another, when stress is applied to the material, thereby providing the required strain relief. In the present embodiment, the plurality of perforations 6018 that form a plurality of curvilinear discontinuous portions 6020 are depicted as curvilinear, however, the plurality of perforations 6018 need not be curvilinear and instead can be any geometry such as, for example, polygonal—square or rectangular, which form correspondingly-shaped discontinuous portions 6020, see for example, discontinuous portions 2870 in FIGS. 2C and 2D. It is only required that the plurality of perforations 6018 result in a plurality of discontinuous portions 6020 being formed in the bottom layer 6006 material that separate from each other and move with the skin, independent of one another.

As illustrated in the figures, the top layer 6004 is attached to the bottom layer 6006 with the first layer adhesive thereby sandwiching the bottom layer 6006 between the top layer 6004 and the skin when the adhesive system 6000 is attached to the skin. In this embodiment, because the perforations 6018 extend through the entire thickness of the bottom layer 6006, which create discontinuous portions 6020 that are adjacent to one another, the bottom layer 6006 typically has a lower effective elastic modulus than the top layer 6004. Therefore, the top layer 6004 provides structural reinforcement for the bottom layer 6004 and holds the adhesive system 6000 together.

As depicted in FIG. 2J, which is a bottom view of the adhesive system 6000, the top layer 6004 has a first perimeter 6022 that defines a first area and the bottom layer 6006 has a second perimeter 6024 that defines a second area. In some embodiments, the first area is greater than the second area, which results in portions 6026 of the first perimeter 6022 extending beyond the second perimeter 6024. Thus, when the adhesive system 6000 is attached to the skin, in addition to the bottom layer 6006 adhering to the skin with the bottom layer adhesive, the portions 6026 of the top layer 6004 that extend beyond the perimeter 6022 of the bottom layer 6006 (i.e., overhang the bottom layer 6006), result in a portion of the top layer 6004 also adhering to the skin with the top layer adhesive. In some embodiments, the bottom layer adhesive can be less aggressive than the top layer adhesive. In the present embodiment, a less aggressive adhesive may be used to adhere the bottom layer 6006 to the skin as the plurality of discontinuous portions 6020 transform the bottom layer into a very low elastic modulus layer. Because the discontinuous portions 6020 separate under low stress and therefore, move with the skin independently of one another, the bottom layer adhesive can be less aggressive as the shear forces between the discontinuous portions 6020 and the skin, are low. The lower shear forces result from the smaller contact area between the bottom layer adhesive on the discontinuous portions 6020 and the skin. Thus, smaller area discontinuous portions 6020 allow less aggressive adhesives to be used resulting in reduced skin irritation and easier and less painful removal from the skin. In this embodiment, the top layer 6004 and the bottom layer 6006, are attached to the skin with an adhesive.

In some embodiments, the top layer adhesive used to attach the top layer 6004 to the bottom layer 6006 and the portions 6026 of the top layer that extend beyond the perimeter 6022 of the bottom layer 6006 to the skin, is a more aggressive adhesive than the bottom layer adhesive. This more aggressive adhesive is necessary to keep the top layer attached to the bottom layer 6006 and the skin when stress is applied to the adhesive system 6000 due to movement (expansion and contraction) of the skin. That is, the top layer 6004 must expand and contract to the same extent as the skin in order to cause the perforations 6008, 6012 to open and preferentially induce formation of the concentrated areas of stress 6016 and hence, plastic deformation of the top layer 6004, thereby minimizing stress in the top layer 6004. Thus, the top layer 6004 must remain attached to the skin.

In addition to using an aggressive adhesive to impart a higher initial and sustained bond strength between the portions 6026 of the top layer 6004 that extend beyond the perimeter 6024 of the bottom layer 6006 that attach to the skin with the top layer adhesive, the area of the portions 6026 of the top layer 6004 that extend beyond the perimeter 6024 of the bottom layer 6006 can be increased such that a larger area of the top layer 6004 is attached to the skin with the top layer adhesive. The increased area of the top layer 6004 that adheres to the skin allows a less aggressive adhesive to be used while keeping the adhesive system 6000 attached to the skin and causing the adhesive system 6000 to plastically deform under the stress imparted due to movement of the skin.

In additional embodiments of a two-layer adhesive system according to the present invention, as depicted in FIGS. 2K and 2L, the adhesive system 6000 includes a top layer 6004, which can be constructed in accordance with embodiments herein to include, for example, a plurality of perforations 6008, along a first direction, and/or a plurality of perforations 6012, along a second direction that create openings in the material and concentrated areas of stress 6016 between adjacent perforations as depicted in FIG. 2I. The bottom layer 6006 can comprise a hydrocolloid. Because hydrocolloids are low elastic modulus materials with high MVTRs, in these embodiments, the bottom layer 6006 may (FIG. 2L) or may not (FIG. 2K) include the plurality of perforations 6004, 6008 therein that the top layer 6004 includes.

Depicted in FIGS. 2M to 2R are additional embodiments of the present multilayer adhesive system. The adhesive systems 6500, 6600 are three-layer systems that include a top layer 6504, 6604, middle layer 6508, 6608 and bottom layer 6512, 6612. The top layer 6504 can be made from a material having an inherent low elastic modulus and an inherent high MVTR or it can be formed of a material that is modified to have an effective lower elastic modulus and/or an effective higher MVTR. The modifications can be, for example, a plurality of perforations 6008 along a first direction, and/or a plurality of perforations 6012 along a second direction that create concentrated areas of stress 6016 between adjacent perforations as depicted in FIG. 2F. In some embodiments, the top layer is a polyurethane material. In some embodiments, the top layer is a silicone elastomer.

In the embodiment depicted in FIG. 2N, the middle layer 6508 can be a separate adhesive to attach the top layer 6505 to the bottom layer 6512. In some embodiments, the middle layer 6508 can be a fiber reinforced adhesive, such as, for example, a polyester fiber reinforced acrylate adhesive. Because fiber reinforced adhesives typically have a higher elastic modulus than desired, as depicted in FIGS. 20 and 2P where FIG. 2P is a bottom view of the adhesive system 6500, the middle layer 6508 in these embodiments can also include the plurality of perforations 6008 along a first direction, and/or the plurality of perforations 6012 along a second direction, similar to the top layer 6504, in order to reduce the elastic modulus of the middle layer 6508. In some embodiments, as depicted in FIG. 2N, the middle layer 6508 is unmodified.

As depicted in FIGS. 2 N to 2P, the bottom layer 6512 can comprise a hydrophobic material or a wicking material such as, for example, a spun lace non-woven material, that includes and adhesive for adhering the bottom layer 6512 to skin. As illustrated in the figures, the bottom layer 6512 in these embodiments, can be constructed in a similar manner with similar properties as the bottom layer 6006 for the two layer embodiments of the present adhesive system (see for example. FIG. 2H), to include a plurality of perforations 6018 therein that form a plurality of discontinuous portions 6020. Accordingly, when the bottom layer 6512 is adhered to skin and is stressed, the plurality of discontinuous portions 6020 separate from each other, thereby providing strain relief in the bottom layer 6512. Because the discontinuous portions 6020 are adhered to the skin, once they separate from the adjacent discontinuous portions 6020, they move with the skin, independently of one another. Thus, the same wicking material designs disclosed above for the bottom layer 6006 of the two-layer adhesive system embodiments, can be used for the three-layer adhesive system embodiments.

In another embodiment of the three-layer adhesive system 6600, as depicted in FIGS. 2Q and 2R, the system includes a top layer 6604, middle layer 6608 and bottom layer 6612. The top layer 6604 can be, similar to previous embodiments, made from a material having an inherent low elastic modulus and an inherent high MVTR or it can be formed of a material that is modified to have an effective lower elastic modulus and/or an effective higher MVTR. The modifications can be, for example, a plurality of perforations 6008 along a first direction, and/or a plurality of perforations 6012 along a second direction that create concentrated areas of stress 6016 between adjacent perforations as depicted in FIG. 2I. In some embodiments, the top layer is a polyurethane material. In some embodiments, the top layer is a silicone elastomer.

In the embodiment depicted in FIG. 2Q, the middle layer 6608 can comprise a hydrophobic material or a wicking material such as, for example, a spun lace non-woven material. As illustrated, the middle layer 6608 in these embodiments, can be constructed in a similar manner with similar properties as the bottom layer 6006 for the two layer embodiments of the present adhesive system depicted in FIG. 2I, to include a plurality of perforations 6018 therein that form a plurality of discontinuous portions 6020. In this embodiment, the bottom layer 6612 can comprise a hydrocolloid, which attaches to the middle layer 6608 and the skin. Accordingly, when the three-layer adhesive system 6600 is adhered to skin and is stressed, the plurality of discontinuous portions 6020 of the middle layer 6608 to move with the hydrocolloid, which moves with the skin because it is a low elastic modulus material, and separate from each other, thereby providing strain relief in the middle layer 6608. Because the discontinuous portions 6020 are adhered to the skin through the hydrocolloid, once they separate from the adjacent discontinuous portions 6020, they move with the skin, independently of one another. Thus, the same wicking material designs disclosed above for the bottom layer 6006 of the two-layer adhesive system embodiments, can be used for the middle layer 6608 in this embodiment of the three-layer adhesive system.

In the three-layer adhesive system embodiments 6500, 6600 depicted in FIGS. 2M-2R, the top layer 6504, 6604 has a first perimeter 6522, 6622 that defines a first area, the middle layer 6508, 6608 has a second perimeter 6524, 6624 that defines a second area and the bottom layer 6512, 6612 has a third perimeter 6526, 6626 that defines a third area. In some embodiments, the first area is greater than the second and third areas, which results in portions 6528, 6628 of the first perimeter 6522, 6622 extending beyond the second and third perimeters 6524, 6624, 6526, 6626 (see FIGS. 2P and 2R). Thus, when the adhesive systems 6500, 6600 are attached to the skin, in addition to the bottom layer 6512, 6612 adhering to the skin, the portions 6528, 6628 of the top layer 6504, 6604 that extend beyond the perimeters 6524, 6624, 6526, 6626 of the middle layer 6508, 6608 and bottom layer 6512, 6612 (i.e., overhang the middle layer 6508, 6608 and bottom layer 6512, 6612), result in a portion of the top layer 6504, 6604 also adhering to the skin. Accordingly, adhesives with similar properties to those disclosed above for the two-layer adhesive system embodiments can be used to attach the three-layer adhesive system embodiments to skin.

As previously disclosed, the length of the perforations 6008, 6012 and the spacing between adjacent perforations in the embodiments of the adhesive systems disclosed herein, can be changed/adjusted to tune the effective elastic modulus of the materials/layers and hence, the effective modulus of the completed multilayer adhesive systems.

As illustrated in FIG. 2S, embodiments of the present adhesive systems can include layers that have been modified to include a plurality of first perforations 6008 along a first direction 6010 and a plurality of second perforations 6012 along a second direction 6014. In some embodiments, (a) the plurality of first perforations 6008 have a length L1 and adjacent first perforations 6008 are separated by a distance L2 and (b) the plurality of second perforations 6012 have a length L3 and adjacent second perforations 6012 are separated by a distance L4. The lengths L1 and L3 and the distances L2 and LA can be chosen to change the size of the concentrated areas of stress 6016 that are created between adjacent first perforations 6008 and adjacent second perforations 6012, which changes the effective elastic modulus of the layer that includes the first and second perforations 6008, 6012. Thus, for example, when L1 and L3 have lengths that are longer than the distances L2 and L4A, the layer will have an effective elastic modulus that is lower than a layer having an L1 and L3 with lengths that are shorter than the distances L2 and LA. Accordingly, adhesive system layer embodiments that include first and second perforations 6008, 6012 having lengths L1 and L3, respectively, that are significantly longer than the distances L2 and LA, will have a much lower elastic modulus than adhesive system layer embodiments that include first and second perforations 6008, 6012 having lengths L1 and L3, respectively, that are not significantly longer than the distances L2 and LA. In some embodiments, L1 is substantially equal to L3 and L2 is substantially equal to LA, which results in a layer/adhesive system having an effective elastic modulus that is substantially the same in both the first and second directions 6010, 6014. In some embodiments, L1 is not substantially equal to L3 and L2 is not substantially equal to L4A, which results in a layer/system having an effective elastic modulus that is not substantially the same in both the first and second directions 6010, 6014. In some embodiments, L1 and L3 can range from approximately 1.0 mm to 3.0 mm and L2 and LA can range from approximately 0.25 mm to 1.0 mm. Also, in some embodiments, adhesive system layers may only include perforations along one direction so as to only substantially change the effective elastic modulus of the layer/material in one direction.

Although the plurality of perforations in the disclosed embodiments are shown in a cross-hatch pattern or are orthogonal to one another, any pattern of a plurality of perforations that create concentrated areas of stress in a layer or multilayer adhesive system, may be used. The type of patterned perforations used will affect the effective elastic modulus of the layer and/or adhesive system.

Modifying L1, L2, L3, and LA as outlined above, allows the effective elastic modulus of an individual layer or the constructed multilayer adhesive system to be tuned/adjusted to be less than approximately 100 Kpa, 90 Kpa, 70 Kpa, 60 Kpa, 50 Kpa, 40 Kpa, 30 Kpa, 20 Kpa, and 10 Kpa, at 100% strain. Thus, modifying the individual layers or the constructed multilayer adhesive system as outlined above, allows the effective elastic modulus to be maintained for strains up to 0.4 and preferably, up to 1.0.

In some embodiments of the two-layer adhesive systems disclosed herein, the top layer can have an effective elastic modulus less than 0.02 Mpa (20 Kpa) that is maintained for strains up to 0.4 and preferably, for strains up to 1.0. In some embodiments, the bottom layer can have an effective elastic modulus less than 0.02 Mpa (20 Kpa) that is maintained for strains up to 0.4 and preferably, for strains up to 1.0. In some embodiments, the two-layer adhesive system can have an effective elastic modulus less than 0.02 Mpa (20 Kpa) that is maintained for strains up to 0.4 and preferably, for strains up to 1.0. In some embodiments, the concentrated areas of stress plastically deform when an external load is applied to achieve a net strain of up to 0.4 in the two-layer adhesive system. In some embodiments, when the multilayer adhesive system is deformed by an external load to a strain of up to 0.4, the multilayer adhesive system deforms resulting in >90% of the achieved strain being retained when the external load is removed.

In some embodiments of the three-layer adhesive systems disclosed herein, the top layer can have an effective elastic modulus less than 0.02 Mpa (20 Kpa) that is maintained for strains up to 0.4 and preferably, for strains up to 1.0. In some embodiments, the middle layer can have an effective elastic modulus less than 0.02 Mpa (20 Kpa) that is maintained for strains up to 0.4 and preferably, for strains up to 1.0. In some embodiments, the bottom layer can have an effective elastic modulus less than 0.02 Mpa (20 Kpa) that is maintained for strains up to 0.4 and preferably, for strains up to 1.0. In some embodiments, the three-layer adhesive system can have an effective elastic modulus less than 0.02 Mpa (20 Kpa) that is maintained for strains up to 0.4 and preferably, for strains up to 1.0. In some embodiments, the concentrated areas of stress plastically deform when an external load is applied to achieve a net strain of up to 0.4 in the two-layer adhesive system. In some embodiments, when the multilayer adhesive system is deformed by an external load to a strain of up to 0.4, the multilayer adhesive system deforms resulting in >90% of the achieved strain being retained when the external load is removed.

Depicted in FIG. 2T is a chart showing the results of strain tests that were performed on adhesive systems constructed in accordance with the embodiments disclosed herein. As used in the description of FIG. 2T, unmodified means that the layer was not modified as disclosed herein to include any perforations therein and modified means that the layer was modified to include either a plurality of perforations in the first and second directions (for the polyurethane (PU) top layer and the adhesive middle layer) or a plurality of perforations that form a plurality of discontinuous portions therein (the adhesive-backed spun lace, non-woven bottom layer). It should be noted that the adhesive systems identified in the chart started to plastically deform at 40% strain, reducing the slope calculation of the modulus.

The following seven adhesive systems were tested. Set 1 comprised an adhesive system having an unmodified polyurethane top layer. At 25% strain, the elastic modulus was approximately 15 Kpa and at 40% strain, the elastic modulus was approximately 14 Kpa. Set 2a comprised an unmodified polyurethane top layer and an unmodified hydrocolloid bottom layer. At 25% strain, the elastic modulus was approximately 15 Kpa and at 40% strain, the elastic modulus was approximately 16 Kpa. Set 2b comprised a modified polyurethane top layer and an unmodified hydrocolloid bottom layer. At 25% strain, the elastic modulus was approximately 10 Kpa and at 40% strain, the elastic modulus was approximately 10 Kpa. Set 3a comprised an unmodified polyurethane top layer and an unmodified adhesive backed spun lace, non-woven bottom layer. At 25% strain, the elastic modulus was approximately 44 Kpa and at 40% strain, the elastic modulus was approximately 38 Kpa. Set 3b comprised an unmodified polyurethane top layer, an unmodified adhesive middle layer and an unmodified adhesive backed spun lace, non-woven bottom layer. At 25% strain, the elastic modulus was approximately 64 Kpa and at 40% strain, the elastic modulus was approximately 51 Kpa. Set 4a comprised a modified polyurethane top layer and a modified adhesive backed spun lace, non-woven bottom layer. At 25% strain, the elastic modulus was approximately 25 Kpa and at 40% strain, the elastic modulus was approximately 0 Kpa. Set 4b comprised a modified polyurethane top layer, a modified adhesive middle layer and a modified adhesive backed spun lace, non-woven bottom layer. At 25% strain, the elastic modulus was approximately 22 Kpa and at 40% strain, the elastic modulus was approximately 19 Kpa.

As can clearly be seen in FIG. 2T. modifying the adhesive layers as disclosed herein, reduces the materials and hence, the adhesive system's elastic modulus.

Depicted in FIGS. 2U to 2W is an illustration of how adhesive systems according to embodiments of the present invention react and respond when attached to skin. FIGS. 2U to 2W are cross-sectional views through a two-layer adhesive system according to embodiments of the present invention, for example, the embodiments associated with FIGS. 2E to 2I. Although a two-layer system adhesive system is depicted, three-layer adhesive systems of the embodiments of the present invention will react and respond in a similar manner.

FIG. 2U depicts the adhesive system 6000 when initially attached to the skin 6001. As can be seen in the figure, the adhesive system 6000 includes a top layer 6004 with a plurality of perforations 6008 along a first direction that is attached to a middle layer 6006 with a top layer adhesive 6005. The bottom layer 6006 attaches to the skin 6001 with a bottom layer adhesive 6007 and includes a plurality of perforations 6018 that form a plurality of discontinuous portions 6020 in the bottom layer 6006.

As depicted in FIG. 2V, when the skin 6001 stretches in the direction indicated by arrows 6021, the discontinuous portions 6020 of the bottom layer 6006, which are attached to the skin 6001 with bottom layer adhesive 6007, also move in direction 6021 causing any discontinuous portions 6020 that are connected to adjacent discontinuous portions 6020 to separate. Accordingly, movement of the discontinuous portions 6020 away from each other causes the material of the top layer 6004, which is attached to the bottom layer 6006 with top layer adhesive 6005, to move in a corresponding manner. This movement imparts stress on the top layer 6004, which causes the concentrated areas of stress 6016 to form in the areas between adjacent perforations 6008 in the top layer 6004. These concentrated areas of stress 6016 plastically deform and elongate under the stress applied by movement of the skin 6001 as a result of the top layer 6004 being stretched beyond its elastic limit. This plastic deformation provides strain relief between the adhesive system 6000 and the skin 6001.

Once the skin 6001 is unstressed or returned to its relaxed state, which is depicted in FIG. 2W, the concentrated areas of stress 6016 in the top layer 6004 that plastically deformed and hence, elongated, now form wrinkles 6025 in the adhesive system 6000. As a result of top layer's 6004 plastic deformation and the discontinuous portions 6020 separating from each other, the shear forces/stress between the skin 6001 and bottom layer adhesive 6007 is reduced. In subsequent movement/stretching of the skin 6001 and the adhesive system 6000, the discontinuous portions 6020 of the bottom layer 6006 and the material of the top layer 6004 can now move freely with the skin as the wrinkles 6025 or elongated material of the top layer 6004, freely elongate allowing the adhesive system 6000 to move with the skin 6001 with very minimal shear forces between the adhesive system 6000 and skin 6001. Thus, there is minimal “pulling” on the adhesive system, which drastically reduces the occurrence of edge peel. If the wrinkled portions 6025 are elongated past there previously deformed length, these wrinkled portions 6025 again undergo plastic deformation and elongate, thereby creating larger wrinkles 6025, which again reduce shear forces between the adhesive system 6000 and skin 6001.

In addition, this reduction in shear forces/stress after plastic deformation, permits the use of an adhesive that has a high initial bond strength with a lower sustained bond strength, which results in adhesive systems that are easy to remove with less pain and that are able to be removed as an intact system (in one piece).

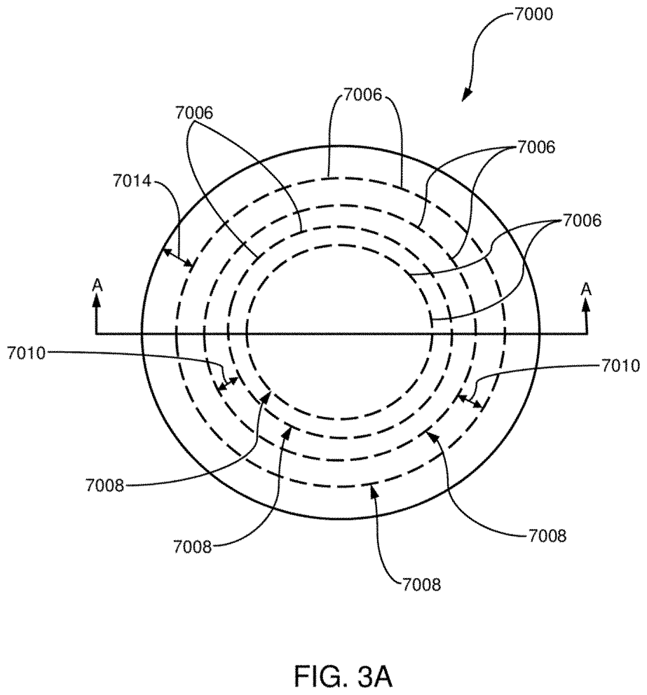

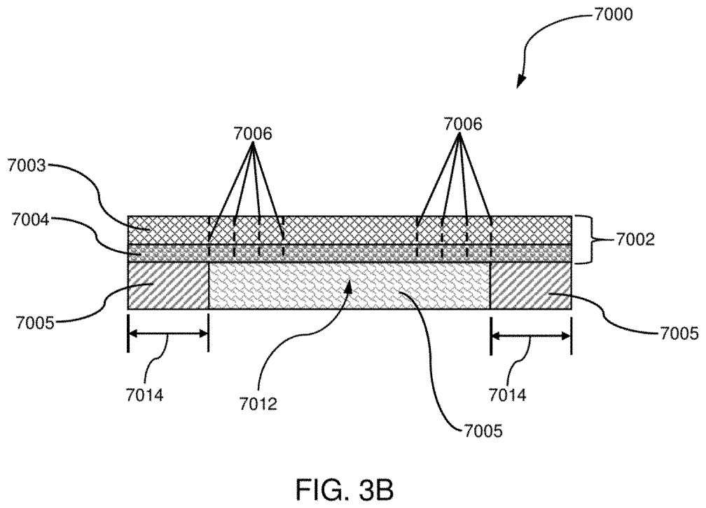

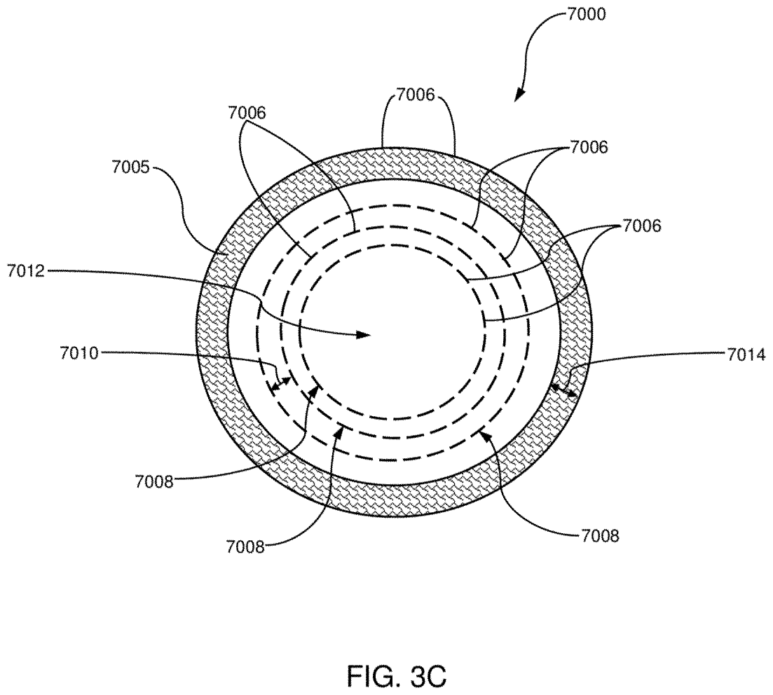

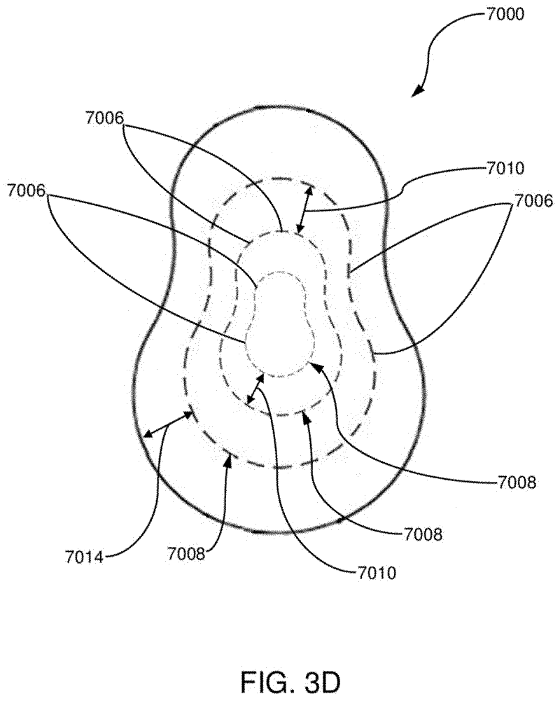

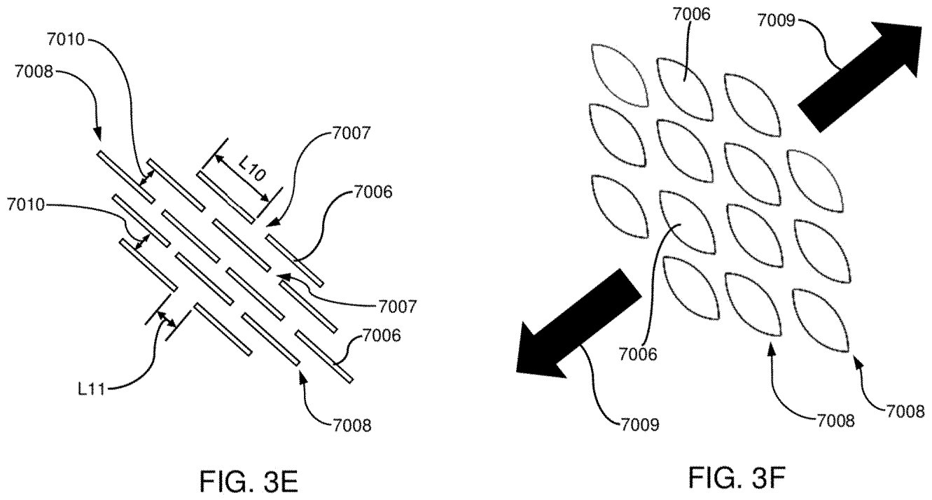

Depicted in FIGS. 3A, 3B and 3C is another embodiment of an adhesive system according to the present invention. In this embodiment, the adhesive system 7000 is depicted as circular but as will be readily understood by those of skill in the art, the adhesive system can be any shape, for example, the shape depicted in FIG. 3D. As depicted in FIGS. 3A, 3B and 3C, where FIG. 3B is a cross-section of the adhesive system depicted in FIG. 3A taken along line A-A in FIG. 3A, the adhesive system 7000 comprises a single layer of material 7002 that either has a low inherent elastic modulus or that is modified (as discussed herein) to have a low effective elastic modulus. Even if the material has a low inherent elastic modulus, the elastic modulus of the material can be further lowered through modification of the material as described herein. In some embodiments, the single layer of material 7002 is a spun lace, non-woven material 7003 that includes an adhesive 7004 thereon for adhering to skin. As will be readily understood by those of skill in the art, the single layer of material 7002 can include any material 7003 that is suitable for the adhesive system's intended use, i.e., suitable to attach a medical device or medical appliance to skin, suitable for wound care and healing, etc.

As can best be seen in FIGS. 3B and 3C, the single layer of material 7002 includes a ring of an additional adhesive 7005 along its bottom perimeter that is more aggressive than the adhesive 7004 included on the single layer of material 7002, i.e., on the interior bottom portion 7012 of the adhesive system. In some embodiments, the more aggressive adhesive 7005 is a hydrocolloid material.

Although, as depicted in FIG. 3B, the additional adhesive 7005 is shown to be placed on top of adhesive 7004, in some embodiments, the additional adhesive 7005 is placed in direct contact with the spun lace, non-woven material 7003. In some embodiments, the adhesives 7004 and 7005 are flush with each other in the interior bottom portion 7012 of the adhesive system 7000.

To lower the effective elastic modulus of the single layer of material 7002, the single layer of material 7002 can be modified, for example, to include a plurality of modifications, such as, for example, a plurality of perforations 7006 that extend through the thickness of the spun lace, non-woven material 7003 and which can also extend through the adhesive 7004. Additionally, in some embodiments, the ring-shaped perforations 7006 may extend completely through the single layer of material 7002 while in other embodiments, they may not extend completely through the thickness of the material/layer 7002 and instead may be recessed, indented or embossed portions that fail when under stress thereby creating the through cut perforations 7006. As disclosed and described with respect to the previous adhesive system embodiments, the perforations 7006 can be any openings, slits, cuts or other perforations that can open up as discussed below.

As can be seen in FIGS. 3A and 3C, the perforations 7006 form a plurality of rings 7008. In the embodiment depicted in FIGS. 3A-3C, there are four (4) rings 7008 of perforations 7006. The number of rings 7008 of perforations 7006 effects the effective elastic modulus of the single layer of material 7002. That is, the greater the number of rings 7008, the lower the effective elastic modulus of the adhesive system. Thus, as will be readily understood to those skilled in the art, the adhesive system 7000 can be constructed to have any number of rings 7008, for example, 1, 2, 3, 4, 5, 6, 7, 8, 9, 10 or more, rings 7008, which permits one to design adhesive systems to have different effective elastic moduli based on the number of rings 7008 included.

In some embodiments, the distance 7010 between all adjacent rings 7008 is the same while in some embodiments, the distance 7010 between adjacent rings 7008 varies. The distance 7010 between adjacent rings 7008 also effects the elastic modulus of the single layer of material 7002. For example, a smaller distance 7010 between adjacent rings 7008 allows more rings 7008 and hence, more perforations 7006, to be included for the adhesive system. The higher the number of rings 7008 that are included, the lower the effective elastic modulus of the system.

The plurality of ring-shaped perforations 7006 transform the single layer of material 7002 from a material having a high or higher elastic modulus and/or a low or lower inherent MVTR into a material having an effective low or lower elastic modulus and/or an effective high or higher MVTR. The effective lower elastic modulus is achieved because when the adhesive system 7000 is attached to skin for example, the strain that is applied to or generated on the adhesive system 7000 as a result of skin movement (stretching/compressing), results in a lower stress on the adhesive system 7000 due to the opening or expansion of the plurality of perforations 7006 as depicted in FIGS. 3E and 3F, where FIG. 3E depicts the perforations 7006 prior to strain 7009 being applied and FIG. 3F depicts the perforations 7006 after strain 7009 is applied to the adhesive system 7000. As can be seen in FIG. 3F, the applied strain 7009 causes the perforations 7006 to “open” or “expand” thereby relieving the stress experienced by the adhesive system 7000, reducing the elastic modulus of the adhesive system 7000, which also reduces the shear forces on the adhesive system.

In addition to the number of rings 7008 of perforations 7006 that are included and the spacing/distance 7010 between adjacent rings 7008 effecting the effective elastic modulus of the single layer of material 7002, as depicted in FIG. 3E, the length L10 of the perforations 7006 and the distance L11 between adjacent perforations 7006 also effect the effective elastic modulus. For example, adhesive systems having perforations 7006 with a longer length L10 allow the perforations 7006 to open or expand to a greater extent under strain, which results in an adhesive system having a lower effective elastic modulus than an adhesive system with perforations 7006 that have a shorter length L10. Additionally, adjacent perforations 7006 that are separated by a shorter distance L11 result in an adhesive system having a lower effective modulus than adhesive systems having adjacent perforations 7006 that are separated by a larger distance L11. In some embodiments, L10 can range from approximately 1.0 mm to 5.0 mm and is preferably 2.0 mm or 3.0 mm or 4.0 mm and L11 can range from approximately 0.25 mm to 3.0 mm and is preferably 1.0 mm or 2.0 mm. In some embodiments, the distance 7010 between adjacent rings 7008 can range between approximately 1.0 mm to 5.0 mm and is preferably 1.0 mm or 2.0 mm.

As depicted in FIGS. 3E and 3F, in some embodiments, the perforations 7006 in one ring 7008 are offset from the perforations 7006 in an adjacent ring 7008. That is, the perforations 7006 and the gaps or distance 7007 between adjacent perforations 7006 in one ring 7008 do not line-up with the perforations 7006 and the gaps 7007 between adjacent perforations 7006 in an adjacent ring.

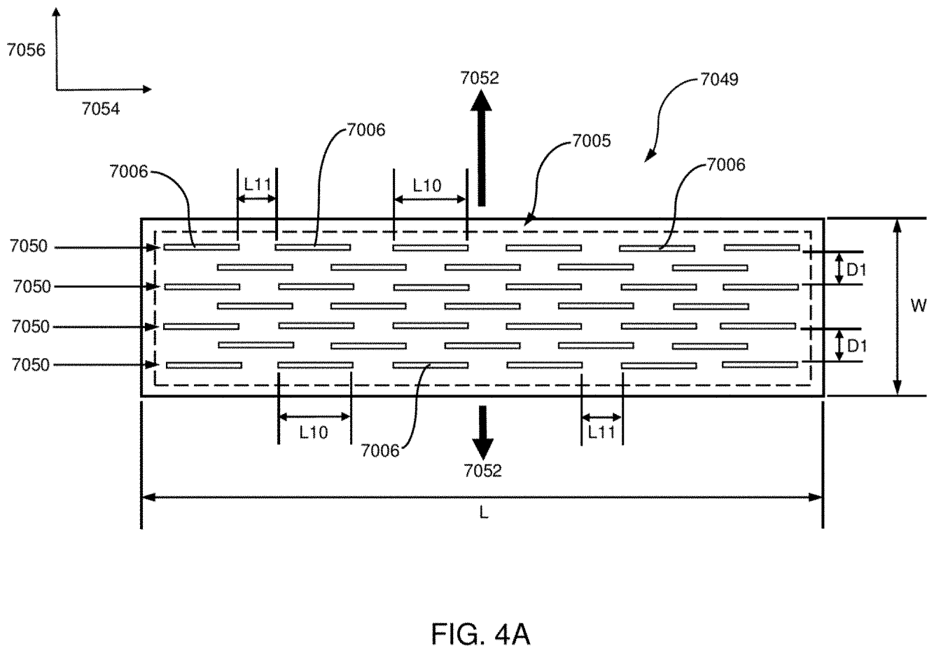

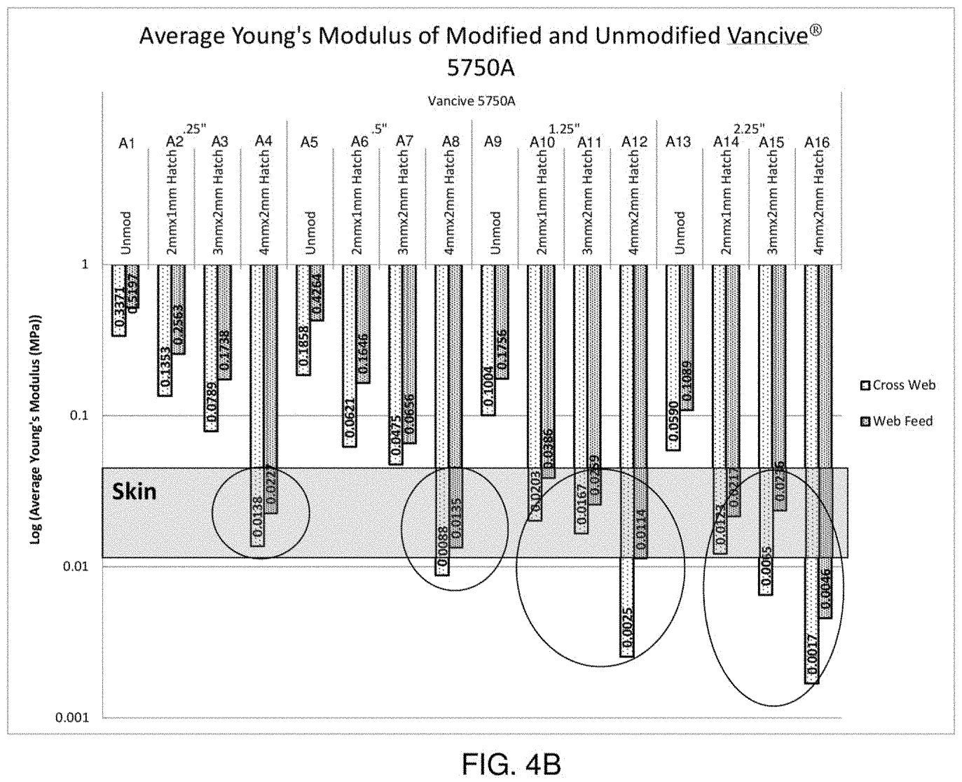

Although the plurality of perforations 7006 in the disclosed embodiments are shown as ring-shaped or circular, any pattern of a plurality of perforations that allow the perforations 7006 to open/expand under strain, may be used. For example, the plurality of perforations 7006 can be arranged in a series of parallel linear rows 7050 where the perforations 7006 in adjacent rows are offset from each other as depicted in FIG. 4A. In some embodiments where the plurality of perforations 7006 are arranged in a series of parallel linear rows 7050, the adhesive system 7049 includes an additional adhesive 7005 along its bottom perimeter that is more aggressive than the adhesive included on the majority of the bottom of the adhesive system 7049. In some embodiments, the more aggressive adhesive 7005 is a hydrocolloid material.

Constructing an adhesive system to have a more aggressive adhesive (hydrocolloid 7005 in some embodiments) along its bottom perimeter than on its interior/inner bottom region 7012, helps the system to resist edge peel resulting in an adhesive system that remains attached to the skin for extended periods of time, for example, 21 days or more. The more aggressive adhesive along the bottom perimeter of the adhesive system helps to prevent edge peel while the interior/inner bottom region 7012, which has been modified to mimic skin mechanics in order to address the strain mismatch between the skin and the adhesive system, decrease the shear forces across the adhesive system, which reduces the need for a more aggressive adhesive on the interior/inner region 7012 to address the shear forces. The aggressiveness of the adhesion along the outer perimeter of the adhesive system can also be controlled by the width 7014 of the more aggressive adhesive 7005. That is, a wider aggressive adhesive 7005 along the bottom perimeter will result in a more aggressive adhesion of the adhesive system along its perimeter, which will further reduce edge peel. Such a design results in an adhesive system that (1) can remain attached to skin for extended periods of time and (2) is less irritating to the wearer and easier and less painful to be removed.

Depicted in FIG. 4B is a chart showing the results of strain tests that were performed on sixteen (16) adhesive systems 7049 constructed in accordance with the embodiments disclosed with respect to FIG. 4A. The tests were performed to identify adhesive systems that best ameliorate the strain mismatch between skin and the adhesive system, resulting in adhesive systems that mimic the mechanical properties of human skin, i.e., have a similar or lower modulus of elasticity than skin where the modulus of elasticity of skin ranges between approximately 0.01 MPa and 0.05 MPa. As used when describing FIG. 4B, unmodified means that the adhesive system was not modified as disclosed herein to include any perforations 7006 therein and, as depicted in FIG. 4A, modified means that the layer/material was modified to include a plurality of perforations 7006 arranged in a series of parallel linear rows 7050 where the length L10 of the perforations 7006, the distance L11 between adjacent perforations 7006 and the distance D1 between adjacent rows 7050 varied between the adhesive systems. In all of the tested adhesive systems included in Table 1, the perforations 7006 were oriented perpendicular/orthogonal to the pull direction 7052 (i.e., the direction of applied strain and i.e., orthogonal to the width “W” of the system or parallel to the length “L” of the system) and the perforations 7006 in adjacent rows were offset from each other as depicted in FIG. 4A. Thus, when pulled/strained, the perforations 7006 “opened” as depicted in FIG. 3E thereby reducing the effective modulus of the adhesive system.

TABLE 1

| | | | | | | | Average Effective | |
|---|---|---|---|---|---|---|---|---|
| Adhesive | Dimensions | | Modifications | | | | Young's Modulus | |
| System | Width “W” | Length “L” | Unmodified | L10 | L11 | D1 | Cross-Web | Web Feed |
| A1 | 6.35 mm | 25.4 mm | XX | 0.0 mm | 0.0 mm | 0.0 mm | 0.337 MPa | 0.519 MPa |
| A2 | 6.35 mm | 25.4 mm | | 2.0 mm | 1.0 mm | 2.0 mm | 0.135 MPa | 0.256 MPa |
| A3 | 6.35 mm | 25.4 mm | | 3.0 mm | 2.0 mm | 2.0 mm | 0.079 MPa | 0.174 MPa |
| A4 | 6.35 mm | 25.4 mm | | 4.0 mm | 2.0 mm | 1.0 mm | 0.014 MPa | 0.023 MPa |
| A5 | 12.7 mm | 25.4 mm | XX | 0.0 mm | 0.0 mm | 0.0 mm | 0.186 MPa | 0.426 MPa |
| A6 | 12.7 mm | 25.4 mm | | 2.0 mm | 1.0 mm | 2.0 mm | 0.062 MPa | 0.165 MPa |
| A7 | 12.7 mm | 25.4 mm | | 3.0 mm | 2.0 mm | 2.0 mm | 0.048 MPa | 0.066 MPa |
| A8 | 12.7 mm | 25.4 mm | | 4.0 mm | 2.0 mm | 1.0 mm | 0.009 MPa | 0.014 MPa |
| A9 | 31.75 mm | 25.4 mm | XX | 0.0 mm | 0.0 mm | 0.0 mm | 0.100 MPa | 0.176 MPa |
| A10 | 31.75 mm | 25.4 mm | | 2.0 mm | 1.0 mm | 2.0 mm | 0.020 MPa | 0.039 MPa |
| A11 | 31.75 mm | 25.4 mm | | 3.0 mm | 2.0 mm | 2.0 mm | 0.017 MPa | 0.026 MPa |
| A12 | 31.75 mm | 25.4 mm | | 4.0 mm | 2.0 mm | 1.0 mm | 0.003 MPa | 0.011 MPa |
| A13 | 57.15 mm | 25.4 mm | XX | 0.0 mm | 0.0 mm | 0.0 mm | 0.059 MPa | 0.109 MPa |
| A14 | 57.15 mm | 25.4 mm | | 2.0 mm | 1.0 mm | 2.0 mm | 0.012 MPa | 0.022 MPa |
| A15 | 57.15 mm | 25.4 mm | | 3.0 mm | 2.0 mm | 2.0 mm | 0.007 MPa | 0.024 MPa |
| A16 | 57.15 mm | 25.4 mm | | 4.0 mm | 2.0 mm | 1.0 mm | 0.002 MPa | 0.005 MPa |

All adhesive systems tested and included in Table 1 were comprised of a single layer of nonwoven material available from Vancive™ Medical Technologies under the name MED 5750A and had a construction similar to that depicted in FIG. 4A except that no aggressive adhesive 7005 was included along the bottom perimeter of the systems. MED 5750A is a single layer of polyethylene nonwoven material that includes an acrylic adhesive. Because the elastic modulus of the MED 5750A material differed in the cross-web and the web feed directions of the material, for the adhesive systems included in Table 1, two sets of material were prepared and tested for each adhesive system. That is, for adhesive system A16 for example, a first sample of the MED 5750A material was cut such that the width “W” of the adhesive system was parallel to the web feed direction 7054 of the material and a second sample of the MED 5750A material was cut such that the width “W” of the adhesive system was orthogonal to the web feed direction or parallel to the cross-web direction 7056 of the material. Both the first sample and the second sample were then modified to include the plurality of perforations 7006 in the configurations outlined in Table 1. Next, both samples were loaded into an Instron machine such that the perforations 7006 in both samples were oriented orthogonal to the pull direction 7052 and the samples were “pulled” or strained in order to measure Young's/elastic modulus of each sample adhesive system. The effective elastic modulus of each sample was then plotted on the chart depicted in FIG. 4B. Thus, as can be seen in FIG. 4B, based on the elastic modulus of the unmodified MED 5750A material, the inherent elastic modulus (which is associated with the “unmodified” samples included as samples A1, A5, A9 and A13 of FIG. 4B) of the MED 5750A material was lower in the web feed direction 7054 than in the cross-web direction 7056.

As can clearly be seen in FIG. 4B, the tested adhesive systems having a wider width “W,” resulted in a lower effective elastic modulus. This results from the wider width adhesive systems (samples A4, A8, A12, A16) having a higher number of rows of perforations 7006 than the narrower width adhesive systems.

Adhesive systems having an elastic modulus (whether inherent or effective) that is either within the elastic modulus range of skin or lower than the elastic modulus of skin, perform better. That is, these adhesive systems experience less edge peel and remain attached to skin for longer periods of time than adhesive systems that have not been designed to ameliorate the strain mismatch between skin and the adhesive system resulting in adhesive systems that mimic the mechanical properties of human skin.

Although, the adhesive systems included in Table 1 demonstrate an effective elastic modulus when the pull direction is orthogonal to the orientation of the perforations, those of skill in the art will readily understand that arranging the perforations in a circular or ring pattern as depicted in FIGS. 3A-3C, results in adhesive systems that more closely mimic the mechanical properties of skin because orienting the perforations as such results in adhesive systems having an elastic modulus that has been modified in multiple directions to compensate for the multiple directions in which the adhesive system will be stretched and compressed when attached to skin.

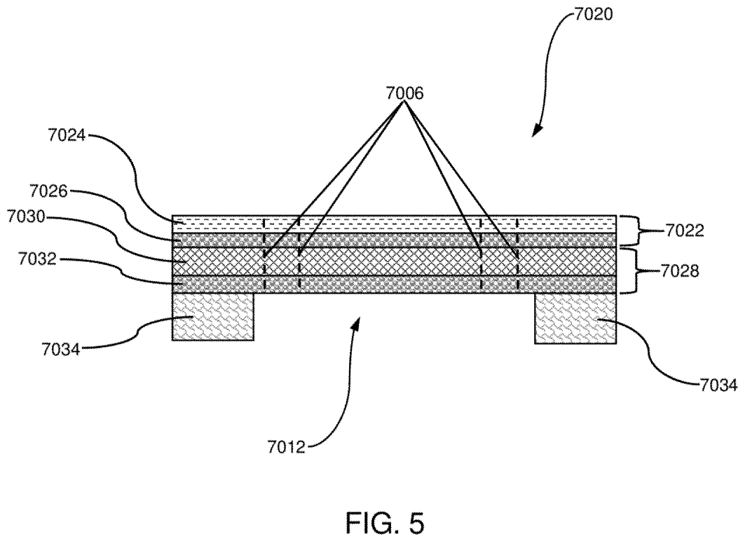

Additional embodiments can include an adhesive system 7020 that includes multiple layers of material having a more aggressive adhesive, such as for example a hydrocolloid, ring around its bottom perimeter, than the adhesive on its interior bottom portion. As depicted in FIG. 5, the adhesive system 7020 can include a top layer 7022 that comprises a first material 7024 and a first adhesive 7026 where the top layer 7022 has an inherent first elastic modulus, a bottom layer 7028 that comprises a second material 7030 and a second adhesive 7032 where the bottom layer has an inherent second elastic modulus, and a third adhesive 7034 along the bottom portion where the third adhesive 7034 is more aggressive than at least the second adhesive 7032. As described above, the top layer 7022 and the bottom layer 7028 can include a plurality of modifications (perforations 7006, for example) therein in order to form an adhesive system that has a low effective elastic modulus. In some embodiments, the first material 7024 is polyurethane, the second material 7030 is a spun lace non-woven material and the third adhesive is a hydrocolloid material. The materials chosen for the top and bottom layers can be chosen based on the desired properties of the adhesive system, i.e., high or low elastic modulus/MVTR. In some embodiments, both layers can include mechanical modifications as discussed above, i.e., a plurality of rings of ring-shaped perforations.

Adhesive systems can be designed to include any number of layers of material with a at least a portion of its bottom surface in contact with skin including a more aggressive adhesive. That is, in some embodiments, the adhesive system can include at least one layer that comprises a single layer of material with an adhesive and a more aggressive adhesive on at least a portion of its bottom surface as disclosed and described with respect to FIGS. 3A-3C. In some embodiments, the adhesive system can include a plurality of layers such as disclosed and described with respect to FIG. 5. In some embodiments, the adhesive system can include 2, 3, 4, 5, 6, 7, 8, 9 or 10 layers of material where each layer can be made from the same materials or different materials having different material and/or mechanical properties (i.e., inherent elastic modulus, fluid wicking properties, fluid absorbing properties, etc.) allowing adhesive systems to be designed for different uses. In some embodiments, adhesive systems having multiple layers of material can be designed to provide a combination of properties (i.e., device attachment properties, wound healing properties, etc.).

In some embodiments, the number of rings 7008 or rows 7050 of perforations 7006, density of perforations 7006, distance 7010/D1 between adjacent rings 7008 or rows 7050 of perforations 7006, length L10 of perforations 7006, and or distance L11 between perforations 7006 in different portions or areas of the same adhesive system, can be adjusted/varied in order to change the effective elastic modulus of different areas/portions of the adhesive system. Thus, adhesive systems can be designed to include multiple effective elastic moduli, which can allow adhesive systems to be designed for attachment to specific areas of the human body.

As previously disclosed, adhesive systems disclosed and described herein, can be used to attach medical devices and medical appliances to the skin. In some examples, the bottom of the device housing 2832 can have channels or other disruptions 2845 that allow air flow under the device housing and also allow moisture to flow away from the skin and adhesive system 6000/7000/7020. The device can therefore be bonded to the underlying adhesive system 6000 in a disrupted manner. The device can be attached to the adhesive system 6000/7000/7020 in a plurality of ways. For example, the device housing 2832 can be attached to the adhesive system 6000/7000/7020 using heat staking, an adhesive layer (e.g. device adhesive 2830 discussed above or any other type of adhesive) or through ultrasonic welding.

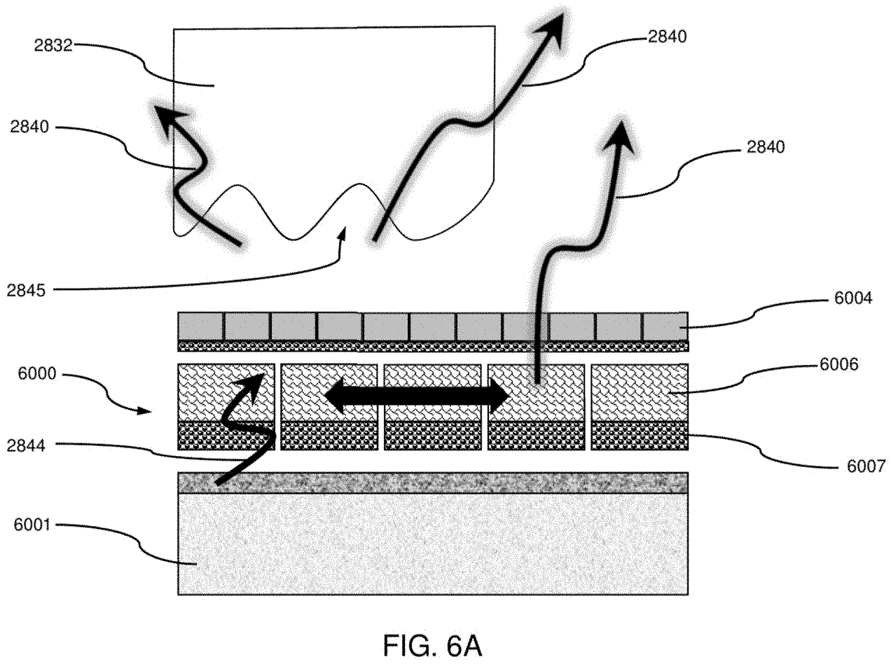

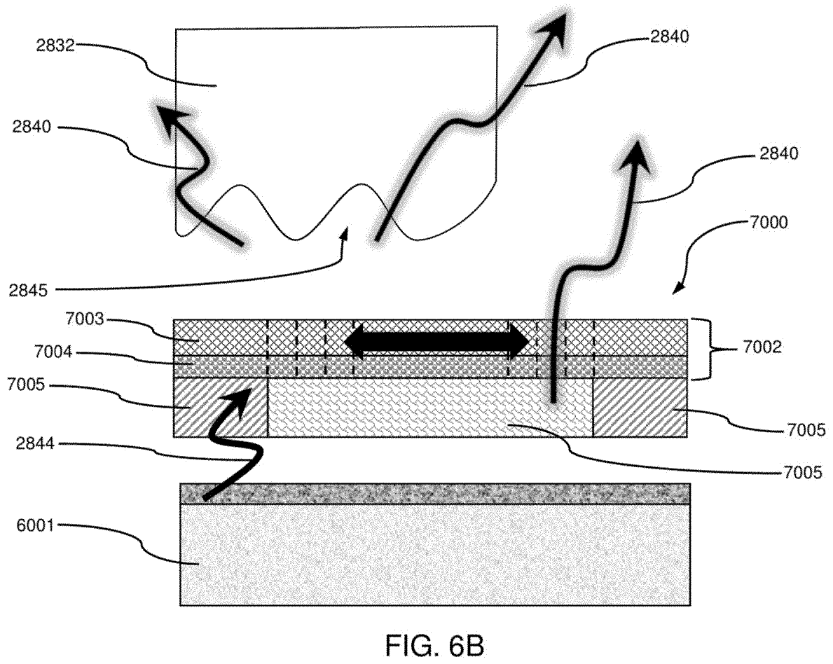

FIGS. 6A and 6B illustrate a schematic view of the device 2832 attached to the skin 6001 with the adhesive system 6000/7000. As discussed above, the material layers of the adhesive system 6000/7000 can provide a high MVTR under the housing of the device 2832 such that water does not accumulate under the device 2832.

FIGS. 6A and 6B include a plurality of arrows that illustrate the movement of moisture from the skin 6001 and through the adhesive system 6000/7000. As denoted by the arrow, the skin 6001 can perspire, generating sweat 2844 that moves to the surface of the skin 6001. The high MVTR material of the adhesive system 6000/7000 can transfer the sweat 2844 the bottom layer 6006/003, which can be a wicking material. The wicking material of the adhesive system 6000/7000 can pull the moisture away from the skin 6001. The adhesive system 6000/7000 can then allow the water vapor 2840 to evaporate from the skin 6001 by causing it to travel laterally through the wicking material of the adhesive system 6000/7000. In some embodiments, the material of the adhesive system 6000/7000 can also serve to repel water from the top surface of the adhesive system 6000/7000. Additionally, any disruptions 2845 on the bottom of the device housing 2832 also helps aid sweat and other water vapor to evaporate from under the adhesive system 6000/7000 and device housing 2832.

Turning briefly to the embodiments of the adhesive systems illustrated in FIGS. 2-5, in some examples, the moisture will wick through the layer of spun lace non-woven material and will evaporate through the top layer, which, in some embodiments, is a modified polyurethane. Evaporation may occur through the plurality of perforations in the top layer of the adhesive systems. In some examples, the moisture will evaporate form the top of the adhesive system and diffuse out from under the sensor housing 2832, 3110 through the disruptions 2845 on the bottom of the sensor housing 2832, 3110.

It is to be understood that the embodiments of the invention described herein are not limited to particular variations set forth herein as various changes or modifications may be made to the embodiments of the invention described and equivalents may be substituted without departing from the spirit and scope of the embodiments of the invention. As will be apparent to those of skill in the art upon reading this disclosure, each of the individual embodiments described and illustrated herein has discrete components and features that may be readily separated from or combined with the features of any of the other several embodiments without departing from the scope or spirit of the embodiments of the present invention. In addition, many modifications may be made to adapt a particular situation, material, composition of matter, process, process act(s) or step(s) to the objective(s), spirit or scope of the embodiments of the present invention. All such modifications are intended to be within the scope of the claims made herein,

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will be understood that when an element is referred to as being “connected” or “coupled” to another element, it can be directly connected or coupled to the other element or intervening elements may be present. In contrast, if an element is referred to as being “directly connected” or “directly coupled” to another element, there are no intervening elements present.

It will be understood that, although the terms first, second, etc. may be used herein to describe various elements, these elements should not be limited by these terms. These terms are only used to distinguish one element from another. Thus, a first element could be termed a second element without departing from the teachings of the present invention.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. Additionally, numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

1.-39. (canceled)

40. An adhesive system, comprising:
a first layer including a first layer material having a top and a bottom having a bottom perimeter, and a first layer adhesive on the bottom for attaching to skin, the first layer having an inherent modulus of elasticity; and
a second adhesive that is more aggressive than the first layer adhesive, the second adhesive along only the bottom perimeter,
wherein the first layer includes a plurality of modifications therein that result in the first layer having an effective modulus of elasticity that is lower than the inherent modulus of elasticity of the first layer.

41. The adhesive system of claim 40, wherein the first layer has a circular shape.

42. The adhesive system of claim 40, wherein the plurality of modifications in the first layer are a plurality of perforations.

43. The adhesive system of claim 42, wherein the plurality of perforations are in a shape of the first layer.

44. The adhesive system of claim 43, wherein the plurality of perforations form a plurality of rows of perforations in the shape of the first layer.

45. The adhesive system of claim 41, wherein the plurality of modifications in the first layer are a plurality of perforations.

46. The adhesive system of claim 45, wherein the plurality of perforations are in the shape of a ring.

47. The adhesive system of claim 45, wherein the plurality of perforations form multiple rings in the first layer.

48. The adhesive system of claim 45, wherein strain applied to the adhesive system causes the plurality of perforations to expand to reduce a stress on the adhesive system that reduces edge peel.

49. The adhesive system of claim 40, wherein the first layer material is a spun lace non-woven material.

50. The adhesive system of claim 40, wherein the second adhesive is a hydrocolloid.

51. The adhesive system of claim 50, wherein the second adhesive is a hydrocolloid.

52. The adhesive system of claim 40, further comprising a second layer comprising:
a second layer material and a second layer adhesive for attaching to the first layer, the second layer having an inherent modulus of elasticity; and
a plurality of modifications therein that result in the second layer having an effective modulus of elasticity that is lower than the second layer's inherent modulus of elasticity

53. An adhesive system, comprising:
a circular spun lace non-woven material comprising a top and a bottom having a bottom perimeter, and an adhesive on the bottom for attaching to skin, the spun lace non-woven material having an inherent modulus of elasticity; and
a hydrocolloid ring around only the bottom perimeter of the spun lace non-woven material,
wherein the spun lace non-woven material includes a plurality of rings comprising a plurality of perforations that transform the adhesive system into an adhesive system having an effective modulus of elasticity that is lower than the inherent modulus of elasticity of the spun lace non-woven material.

54. An adhesive system, comprising:
at least one layer comprising:
a material having a top and a bottom having a bottom perimeter,
a first adhesive on the bottom for at least partially attaching to skin;
a first inherent modulus of elasticity;
a plurality of modifications therein that result in the first layer having an effective modulus of elasticity that is lower than the first inherent modulus of elasticity; and
a second adhesive that is more aggressive than the first adhesive, the second adhesive along only the bottom perimeter.

55. The adhesive system of claim 54, wherein the plurality of modifications are a plurality of perforations.

56. The adhesive system of claim 54, wherein the material is a spun lace non-woven material.

57. The adhesive system of claim 54, wherein the second adhesive is a hydrocolloid.

58. An adhesive system, comprising:
a top layer having a top layer material and a top layer adhesive;
a bottom layer having a bottom layer material with a first side and a second side and a bottom layer adhesive on the second side;
a plurality of layers between the top layer and the bottom layer, each of the plurality of layers having a layer material and a layer adhesive; and
an aggressive adhesive that is more aggressive than the bottom layer adhesive, the aggressive adhesive disposed upon only a portion of the second side of the bottom layer, the aggressive adhesive for attaching to skin,
wherein at least one of the top layer, the bottom layer and the plurality of layers includes a plurality of modifications therein that result in said layer having an effective modulus of elasticity that is lower than the inherent modulus of elasticity of said layer.

59. The adhesive system of claim 58, wherein the plurality of modifications are a plurality of perforations.
